(19) United States
(12) Patent Application Publication
(10) Pub. No.: US 2025/0034603 A1
(43) Pub. Date: Jan. 30, 2025

(54) HEME-CONTAINING CELL CULTURE MEDIA AND USES THEREOF

(71) Applicant: Calysta, Inc., San Mateo, CA (US)

(72) Inventors: Celine Schiff-Deb, San Mateo, CA (US); Lorraine Joan Giver, San Mateo, CA (US); Renee Michelle Saville, San Mateo, CA (US); Lisa Marie Newman, San Mateo, CA (US); Warren Kwan, San Mateo, CA (US); Chien-Seng Hwang, San Mateo, CA (US); Eric Luning, San Mateo, CA (US)

(21) Appl. No.: 18/915,934

(22) Filed: Oct. 15, 2024

Related U.S. Application Data
(63) Continuation of application No. 17/505,411, filed on Oct. 19, 2021.
(60) Provisional application No. 63/094,250, filed on Oct. 20, 2020.

(51) Int. Cl. C12P 7/6432; A23B 4/22; A23C 9/123; A23L 13/00; A23L 17/00; A23L 31/10; A23L 33/135; C12N 1/12; C12N 1/14; C12N 1/16; C12N 1/20; C12N 5/07; C12P 7/6434

(52) U.S. Cl. CPC C12P 7/6432; A23B 4/22; A23C 9/123; A23L 13/00; A23L 17/00; A23L 31/10; A23L 33/135; C12N 1/12; C12N 1/14; C12N 1/16; C12N 1/20; C12N 5/0601; C12P 7/6434; A23V 2002/00; C12N 2511/00

(57) ABSTRACT

The present disclosure provides culture or fermentation media including a biomass or derivative thereof of a hemoprotein-producing C₁ metabolizing non-photosynthetic bacterium, methods of culturing cells or tissue with the culture or fermentation medium, and products produced by the culturing methods including food products, food ingredients, phytoprotective bacterial cell products, and other products of interest such as vitamins, fatty acids, amino acid, carotenoids, etc.

### BACKGROUND

Fermentation and cell or tissue culture involve culturing cells or tissue in a growth medium that includes nutrients needed for survival or growth of the cells. Yeast extracts, peptones and animal serum are commonly used as a source of nutrients for fermentation and cell and tissue culture. However, these sources of nutrients can be cost limiting or deficient in some nutrients to promote optimal growth of the cells. Other nutrient sources for fermentation and cell or tissue culture are needed to support efficient commercial processes.

### SUMMARY

The present disclosure provides cell or tissue culture or fermentation media and ingredients for the same, including a biomass or derivative thereof of a hemoprotein-producing C₁ metabolizing non-photosynthetic bacterium, methods of using the culture or fermentation medium, and products such as food ingredients that are produced from the methods of using the culture or fermentation media.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph of molecular weight distributions of 4 autolysate samples (samples B135, B139, B140 and B141) produced from a biomass of a hemoprotein-producing C₁ metabolizing non-photosynthetic bacterium (Methylococcus capsulatus Bath) according to Example 1. Sample B145 is a sample of biomass homogenate that has not undergone autolysis.

FIG. 2 is a graph of cell growth as measured by spectrophotometry for two marine dwelling bacterial species, Moritella marina and Shewanella pneumatophori, and a marine algae, Schizochytrium sp. ATCC 20888, each cultured in a culture medium without a biomass of a hemoprotein-producing C₁ metabolizing non-photosynthetic bacterium (Methylococcus capsulatus Bath) or an autolysate produced from the biomass, with the biomass (“Biomass Media”), or with an autolysate produced from the biomass (“Autolysate Media”). See Example 2.

FIG. 3 is a graph of concentrations of eicosapentaenoic acid (EPA—C20:5(n−3)) and docosahexaenoic acid (DHA-C22:6(n−3)) produced from cultures of Moritella marina, Shewanella pneumatophori, and Schizochytrium sp. ATCC 20888, each cultured in a culture medium without a biomass of M. capsulatus Bath or an autolysate produced from the biomass, with the biomass (“Biomass Media”), or with an autolysate produced from the biomass (“Autolysate Media”) as well as cell growth of such marine dwelling organisms. See Example 3.

FIGS. 4A and 4B are graphs of Bacillus licheniformis (ATCC 53757) A): cell growth as measured by cultivation in a BioLector cultured in a culture medium without a biomass of a hemoprotein-producing C₁ metabolizing non-photosynthetic bacterium (Methylococcus capsulatus Bath) (“base”) or an autolysate produced from the biomass (“B199”) or with an autolysate produced from the biomass with an added protease (“B223”) and B): Dissolved oxygen traces of the cultivations in A). The graphs represent duplicate cultures grown with and without autolysates at the highest concentration (0.1 g/L N). See Example 4.

FIG. 5 is a graph of Pichia jadini (CBS 4511) cell growth as measured by cultivation in a BioLector cultured in a culture medium without a biomass of a hemoprotein-producing C₁ metabolizing non-photosynthetic bacterium (Methylococcus capsulatus Bath) (“base”) or an autolysate produced from the biomass (“B199”) or with an autolysate produced from the biomass with an added protease (“B223”). The graph represents duplicate cultures grown with and without autolysates at the highest concentration (0.1 g/L N). See Example 4.

FIG. 6 is a graph of Lactobacillus reuteri (DSM 20053) cell growth as measured by cultivation in a BioLector cultured in a culture medium without a biomass of a hemoprotein-producing C₁ metabolizing non-photosynthetic bacterium (Methylococcus capsulatus Bath) (“MRS”) or an autolysate produced from the biomass (“B199”) or with an autolysate produced from the biomass with an added protease (“B223”). The graph represents duplicate cultures grown with and without autolysates at the highest concentration (1.0 g/L N). See Example 4.

FIG. 7 is a graph of Escherichia coli (ATCC 25922) cell growth as measured by cultivation in a BioLector cultured in a culture medium without a biomass of a hemoprotein-producing C₁ metabolizing non-photosynthetic bacterium (Methylococcus capsulatus Bath) (“base”) or an autolysate produced from the biomass (“B199”) or with an autolysate produced from the biomass with an added protease (“B223”). The graph represents duplicate cultures grown with and without autolysates at the highest concentration (0.22 g/L N). See Example 4.

### DETAILED DESCRIPTION

The instant disclosure provides cell or tissue culture or fermentation media and ingredients therefor, including a biomass or derivative thereof of a hemoprotein-producing C₁ metabolizing non-photosynthetic bacterium, methods of using the culture or fermentation media, and products such as food ingredients that are produced from the methods of using the culture or fermentation media.

The biomass or derivative thereof of a hemoprotein producing C1 metabolizing non-photosynthetic bacterium, including homogenates, extracts, lysates, autolysates, and digestates, may be used as an ingredient of a defined or complex growth or culture media to culture or ferment various types of cells or tissue. Examples of cell or tissue types that may be fermented or cultured using the ingredients described herein include bacteria, yeast, fungi, microalgae, mushrooms, animals including insects, and plants including microalgae. The cell or tissue types that are fermented or cultured using the ingredients described herein may be useful for products such as human foods, animal feed, cosmetics, pharmaceuticals and phytoprotective agricultural products. For example, the products may include cell-based meats and meat alternative products (produced from non-animal biomasses), amino acids, peptides, proteins, fatty acids, organic acids, enzymes, pigments, flavors, fragrances, ferments, cultures, probiotics, food ingredients, active ingredients for cosmetics and pharmaceuticals, nucleosides, vitamins, small molecules, metabolites, etc.

The biomass or derivative thereof of the hemoprotein-producing C₁ metabolizing non-photosynthetic bacterial can be used to replace other common nitrogen and/or carbon sources for culture media, especially in complex media, such as peptones, yeast extracts, soytone, and corn meal. One advantage of using the hemoprotein-producing C₁ metabolizing non-photosynthetic bacterial biomass and biomass derivatives (e.g., autolysates, isolates, digestates, extracts, homogenates) as a nitrogen and/or carbon source in culture media is that it imparts a “meat-like” color, a “meat-like” flavor, or other desirable characteristics such as elevated iron levels to cells cultured in the media. The bacterial biomass (or its derivatives) can be used in the culture media to provide heme-containing proteins (i.e., hemoproteins), amino acids and other nutrients (e.g. minerals such as copper, iron) to the cell culture. The hemoproteins present in the C₁ metabolizing non-photosynthetic bacterial biomass (or its derivatives) are delivered in the growth media to cells cultured in the growth media. Biomasses of cells cultured in the growth media have improved meat-like qualities (e.g., red color, metallic flavor, umami flavor, elevated levels of iron), which is useful for production of meat and fish alternative food products. An additional advantage of the culture media ingredient provided herein may include enhanced growth rates, yield, productivity and/or efficiency of the cultured cells or tissue or of the final product of interest, due to essential amino acids, iron, copper, and other nutrients provided by the culture media. This is especially relevant for production systems using fermentation to produce ingredients of interest for the food, feed, cosmetic and phytoprotection sectors, such as meat alternatives, amino acids, peptides and proteins, fatty acids, organic acids, enzymes, pigments, flavors and fragrances, biomass (as ferment, culture, probiotics), active ingredients, etc.

Prior to setting forth this disclosure in more detail, it may be helpful to an understanding thereof to provide definitions of certain terms to be used herein. Additional definitions are set forth throughout this disclosure.

In the present description, the term “about” means ±20% of the indicated range, value, or structure, unless otherwise indicated. The term “consisting essentially of” limits the scope of a claim to the specified materials or steps and those that do not materially affect the basic and novel characteristics of the claimed invention. It should be understood the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof. Any ranges provided herein include all the values and narrower ranges in the ranges.

### A. Cell or Tissue Culture or Fermentation Media

Described herein are cell or tissue culture media, comprising biomass or derivative thereof of a hemoprotein-producing C₁ metabolizing non-photosynthetic bacterium.

The term “culture medium,” “growth medium,” “cell or tissue culture medium,” or “cell or tissue culture or fermentation medium” is a liquid or gel designed to support the growth of microorganisms (e.g., bacteria, yeast, fungi, and microalgae), cells (microbial or derived from multicellular organisms such as animals, insects and plants), tissues or small plants. Culture media generally comprise an appropriate source of energy and nutrients (e.g., carbon source, nitrogen, minerals). In addition to providing nutrients, the medium also helps maintain pH and osmolality in the culture.

“Hemoprotein-producing” refers to the ability of a bacterium to produce one or more hemoproteins. Hemoproteins (also referred to as “heme proteins”) are proteins that are linked to a heme group. Heme is a coordination complex of an iron ion coordinated to a porphyrin molecule. Examples of hemoproteins include hemoglobin, leghemoglobin, myoglobin, cytochromes, catalases, heme peroxidase, and endothelial nitric oxide synthase. Hemoproteins typically contain at least one heme that is tightly bound in stoichiometric amount to the (e.g., with a binding constant in the range of 10⁻⁸ to 10⁻¹⁵ M), and can often be identified by their red color. Hemoproteins may be measured by measuring a peak at 410-415 nm and a peak at 500-550 nm via UV-visible absorption spectroscopy.

As used herein, the term “C₁ substrate” refers herein to any carbon-containing molecule that lacks a carbon-carbon bond. Examples include methane, methanol, formaldehyde, formic acid, carbon monoxide, carbon dioxide, a methylated amine (such as, for example, methyl-, dimethyl-, and trimethylamine), methylated thiols, methyl halogens (e.g., bromomethane, chloromethane, iodomethane, dichloromethane), cyanide, or the like.

As used herein, the term “C₁ metabolizing bacterium” refers to a non-photosynthetic bacterium capable of utilizing C₁ substrates, such as methane, natural gas, biogas, syngas, or unconventional natural gas, as its primary or sole carbon and energy source. In addition, C₁ metabolizing bacteria include “obligate C₁ metabolizing bacteria” that can only utilize C₁ substrates (e.g., methane) for carbon and energy sources, and do not utilize organic compounds that contain carbon-carbon bonds (i.e., multicarbon-containing compounds) as a source of carbon and energy. Also included are “facultative C₁ metabolizing bacteria” that are naturally able to use, in addition to C₁ substrates (e.g., methane), multi-carbon substrates, such as acetate, pyruvate, succinate, malate, or ethanol, as their carbon and energy source.

“Non-photosynthetic” refers to an inability to perform photosynthesis.

In certain embodiments, the C₁ metabolizing non-photosynthetic bacterium is a methylotrophic bacterium.

As used herein, “methylotroph” or “methylotrophic bacteria” refers to a bacterium that is capable of oxidizing organic compounds containing no carbon-carbon bonds, such as methane, methanol, or both. Methylotrophic bacteria include both gram-negative and gram-positive genera. The methylotrophic bacteria of the present disclosure may be aerobic methylotrophic bacteria or anaerobic methylotrophic bacteria. In certain embodiments, a methylotrophic bacterium of the present disclosure is aerobic.

Methylotrophic bacteria include facultative methylotrophs, which have the ability to oxidize organic compounds that do not contain carbon-carbon bonds (e.g., methanol), but may also utilize other carbon substrates such as sugars and complex carbohydrates, and obligate methylotrophs, which are limited to the use of organic compounds that do not contain carbon-carbon bonds. In certain embodiments, a methylotrophic bacterium is an obligate methylotroph. Illustrative obligate methylotrophs include Methylophilus sp., Methylobacillus sp., Methylovorus sp., and Methylophaga sp.

In any of the aforementioned embodiments, a C₁ metabolizing bacterium of this disclosure comprises particular genera of bacterial methylotrophs, such as Methylophilus, Methylopila, Methylobacillus, or Methylobacterium. Examples of methylotrophic bacteria include Methylococcus capsulatus, Methylobacterium extorquens, Methylobacterium radiotolerans, Methylobacterium populi, Methylobacterium chloromethanicum, Methylobacterium nodulans, Methylomonas clara, and Methylobacillus flagellates.

“Methanotrophic bacteria” refers to any methylotrophic bacteria that have the ability to oxidize methane as its primary source of carbon and energy.

In certain embodiments, the C₁ metabolizing bacterium is a methanotrophic bacterium. Methanotrophic bacteria are classified into three groups based on their carbon assimilation pathways and internal membrane structure: type I (gamma proteobacteria), type II (alpha proteobacteria, and type X (gamma proteobacteria). Type I methanotrophs, such as Methylococcus capsulatus, use the ribulose monophosphate (RuMP) pathway for biomass synthesis and generates biomass entirely from CH₄, whereas a Type II methanotroph uses the serine pathway that assimilates 50-70% of the cell carbon from CH₄ and 30-50% from CO₂ (Hanson and Hanson, 1996). Type X methanotrophs use the RuMP pathway but also express low levels of enzymes of the serine pathway.

Methanotrophic bacteria are grouped into several genera, including Methylomonas, Methylobacter, Methylococcus, Methylocystis, Methylosinus, Methylomicrobium, Methanomonas, and Methylocella.

In particular embodiments, the methanotrophic bacterium is selected from the group consisting of Methylomonas, Methylobacter, Methylococcus, Methylosinus, Methylocystis, Methylomicrobium, Methanomonas, and Methylocella.

Methanotrophic bacteria include obligate methanotrophs, which can only utilize C₁ substrates for carbon and energy sources, and facultative methanotrophs, which naturally have the ability to utilize some multi-carbon substrates as a sole carbon and energy source. Facultative methanotrophs include some species of Methylocella, Methylocystis, and Methylocapsa (e.g., Methylocella silvestris, Methylocella palustris, Methylocella tundrae, Methylocystis daltona strain SB2, Methylocystis bryophila, Methylocapsa aurea KYG), and Methylobacterium organophilum (ATCC 27,886). Obligate methanotrophic bacterium are known to produce hemoproteins, such as cytochrome c, cytochrome a, cytochrome b, cytochrome P450, cytochrome c oxidase, catalase, and peroxidase

Exemplary methanotrophic bacteria species include: Methylococcus capsulatus Bath strain, Methylomonas 16a (ATCC PTA 2402), Methylosinus trichosporium OB3b (NRRL B-11,196), Methylosinus sporium (NRRL B-11,197), Methylocystis parvus (NRRL B-11,198), Methylomonas methanica (NRRL B-11,199), Methylomonas albus (NRRL B-11,200), Methylobacter capsulatus (NRRL B-11,201), Methylobacterium organophilum (ATCC 27,886), Methylomonas sp. AJ-3670 (FERM P-2400), Methylocella silvestris, Methylocella palustris (ATCC 700799), Methylocella tundrae, Methylocystis daltona strain SB2, Methylocystis bryophila, Methylocapsa aurea KYG, Methylacidiphilum infernorum, Methylacidiphilum fumariolicum, Methyloacida kamchatkensis, Methylibium petroleiphilum, and Methylomicrobium alcaliphilum.

In certain embodiments, methanotrophic bacteria are aerobic methanotrophic bacteria or anaerobic methanotrophic bacteria. In particular embodiments, methanotrophic bacteria are aerobic methanotrophic bacteria. Aerobic methanotrophs can metabolize methane through a specific enzyme, methane monooxygenase (MMO).

In further embodiments, the methanotrophic bacterium is Methylococcus (e.g., Methylococcus capsulatus, including the strain Methylococcus capsulatus Bath) or Methylosinus (e.g., Methlosinus trichosporium, including the strain Methlosinus trichosporium OB3b).

In particular embodiments, the C₁ metabolizing non-photosynthetic bacterium is Methylococcus capsulatus. The Methylococcus capsulatus of the cell or tissue culture medium may be genetically modified or non-genetically modified. In particular embodiments, the Methylococcus capsulatus is derived from Methylococcus capsulatus (Bath), Methylococcus capsulatus (Texas), Methylococcus capsulatus (Aberdeen), or a combination thereof. In a preferred embodiment, Methylococcus capsulatus of the cell or tissue culture medium includes Methylococcus capsulatus (Bath).

In particular embodiments, the cell or tissue culture or fermentation medium comprises a methanotrophic bacterium and one or more heterologous non-methanotrophic bacteria. For example, a methanotrophic bacterium (e.g., Methylococcus capsulatus Bath) may be cultured with Cupriavidus sp., Anuerinibacillus danicus, or both and optionally in combination with Brevibacillus agri.

In particular embodiments, the C₁ metabolizing non-photosynthetic bacterium of the culture or fermentation medium is non-genetically modified.

In particular embodiments, the C₁ metabolizing non-photosynthetic bacterium comprises a modified C₁ metabolizing bacterium, wherein the modified C₁ metabolizing bacterium comprises at least one recombinant or heterologous polynucleotide that encodes a desired protein, modifies expression of an endogenous protein, or both. In particular embodiments, a recombinant or heterologous polynucleotide encoding a desired protein is operably linked to a promoter. A recombinant or heterologous polynucleotide that modifies expression of an endogenous protein may correspond to an endogenous, heterologous or synthetic regulatory element that controls expression of the endogenous protein, or it may encode a metabolic pathway enzyme whose expression results in the attenuation of expression of the endogenous protein, or the like.

A heterologous or recombinant nucleic acid molecule may be inserted into a C₁ metabolizing non-photosynthetic bacterium means transfected, transduced, transformed, electroporated, or introduction by conjugation (collectively “transformed”), wherein the nucleic acid molecule is incorporated into the genome of the cell, is extra-genomic, is on an episomal plasmid, or any combination thereof.

As used herein, the term “transformation” refers to the process of transferring a nucleic acid molecule (e.g., exogenous or heterologous nucleic acid molecule) into a host cell, which includes all methods of introducing polynucleotides into cells (such as transformation, transfection, transduction, electroporation, introduction by conjugation, or the like). The transformed host cell may carry the exogenous or heterologous nucleic acid molecule extra-chromosomally or the nucleic acid molecule may integrate into the chromosome. Integration into a host genome and self-replicating vectors generally result in genetically stable inheritance of the transformed nucleic acid molecule. Host cells containing the transformed nucleic acids are referred to as “modified,” “recombinant,” “non-naturally occurring,” “genetically engineered,” “transformed” or “transgenic” cells (e.g., bacteria).

Bacterial conjugation, which refers to a particular type of transformation involving direct contact of donor and recipient cells, is frequently used for the transfer of nucleic acids into methanotrophic bacteria. Bacterial conjugation involves mixing “donor” and “recipient” cells together in close contact with each other. Conjugation occurs by formation of cytoplasmic connections between donor and recipient bacteria, with unidirectional transfer of newly synthesized donor nucleic acid molecules into the recipient cells. A recipient in a conjugation reaction is any cell that can accept nucleic acids through horizontal transfer from a donor bacterium. A donor in a conjugation reaction is a bacterium that contains a conjugative plasmid or mobilized plasmid. The physical transfer of the donor plasmid can occur through a self-transmissible plasmid or with the assistance of a “helper” plasmid. Conjugations involving methanotrophic bacteria have been previously described in Stolyar et al., Mikrobiologiya 64:686, 1995; Motoyama et al., Appl. Micro. Biotech. 42:67, 1994; Lloyd et al., Arch. Microbiol. 171:364, 1999; PCT Pub. No. WO 02/18617; and Ali et al., Microbiol. 152:2931, 2006, the methods of which are incorporated by reference herein.

As previously described, the culture or fermentation medium comprises a biomass or derivative thereof of a C₁ metabolizing non-photosynthetic bacterium. The biomass may be derived from whole and/or lysed cells of the C₁ metabolizing non-photosynthetic bacterium. Furthermore, the biomass can be further processed to produce homogenates, extracts, lysates, autolysates, isolates, digestates.

“Biomass” or “bacterial biomass” refers to organic material collected from bacterial culture. Biomass primarily (i.e., more than 50% w/w) comprises bacterial cells, but may include other materials such as lysed bacterial cells, bacterial cell membranes, inclusion bodies, and extracellular material (e.g., products secreted or excreted into the culture medium), or any combination thereof that are collected from bacterial fermentation along with bacterial cells. Preferably, the biomass includes more than 60%, 70%, 75%, 80%, 85%, 90% or 95% cells collected from bacterial fermentation.

To produce a biomass derived from whole and/or lysed cells of the C₁ metabolizing non-photosynthetic bacterium, the bacterium may be cultured with a C₁ substrate under a variety of culture conditions.

As used herein, the term “culturing” or “cultivation” refers to growing a population of cells under suitable conditions in a liquid or a solid medium. Depending on its context, this term may refer to (a) growing C₁ metabolizing non-photosynthetic bacteria to generate a biomass or derivative thereof to be included as an ingredient of a culture or fermentation medium, or (b) growing cells or tissue in a culture or fermentation medium that comprises biomass or derivative thereof of C₁ metabolizing non-photosynthetic bacteria.

In some embodiments, culturing refers to fermentative bioconversion of a C₁ substrate by a C₁ metabolizing non-photosynthetic bacterium into an intermediate or an end product, such as an ingredient for use in a cell or tissue culture or fermentation medium. This culturing step may also be referred to as “culturing to produce a culture media biomass” or the like.

In further embodiment, the C₁ substrate or carbon feedstock is selected methane, methanol, syngas, natural gas, biogas, or combinations thereof. More typically, a carbon feedstock is selected from methane or natural gas. In certain embodiments, culture media may comprise a single C₁ substrate as the sole carbon source for a methanotrophic bacterium, or may comprise a mixture of two or more C₁ substrates (mixed C₁ substrate composition) as multiple carbon sources for a methanotrophic bacterium.

When culturing C₁ metabolizing non-photosynthetic bacteria to produce a growth media biomass is performed in a liquid culture medium, the gaseous C₁ substrates may be introduced and dispersed into a liquid culture medium using any of a number of various known gas-liquid phase systems as described in more detail herein below. When culturing C₁ metabolizing non-photosynthetic bacteria to produce a growth media biomass is performed on a solid culture medium, the gaseous C₁ substrates are introduced over the surface of the solid culture medium.

A variety of culture methodologies may be used for culturing the C₁ metabolizing non-photosynthetic bacterium as described herein. For example, the C₁ metabolizing non-photosynthetic bacterium may be grown by batch culture or continuous culture methodologies. In certain embodiments, the cultures to produce a growth media biomass are grown in a controlled culture unit, such as a fermenter, bioreactor, hollow fiber membrane bioreactor, or the like. Other suitable methods include classical batch or fed-batch culture or continuous or semi-continuous culture methodologies. In certain embodiments, the cultures to produce a growth media biomass are grown in a controlled culture unit, such as a fermenter, bioreactor, hollow fiber membrane bioreactor, and the like.

A classical batch culturing method is a closed system where the composition of the media is set at the beginning of the culture and not subject to external alterations during the culture process. Thus, at the beginning of the culturing process, the media is inoculated with the desired methanotrophic bacteria and growth or metabolic activity is permitted to occur without adding anything further to the system. Typically, however, a “batch” culture is batch with respect to the addition of the methanotrophic substrate and attempts are often made at controlling factors such as pH and oxygen concentration. In batch systems, the metabolite and biomass compositions of the system change constantly up to the time the culture is terminated. Within batch cultures, cells moderate through a static lag phase to a high growth logarithmic phase and finally to a stationary phase where growth rate is diminished or halted. If untreated, cells in the stationary phase will eventually die. Cells in logarithmic growth phase are often responsible for the bulk production of end product or intermediate in some systems. Stationary or post-exponential phase production can be obtained in other systems.

The Fed-Batch system is a variation on the standard batch system. Fed-Batch culture processes comprise a typical batch system with the modification that the methanotrophic substrate is added in increments as the culture progresses. Fed-Batch systems are useful when catabolite repression is apt to inhibit the metabolism of the cells and where it is desirable to have limited amounts of the C₁ substrate in the media. Measurement of the actual substrate concentration in Fed-Batch systems is difficult and is therefore estimated on the basis of the changes of measurable factors, such as pH, dissolved oxygen, and the partial pressure of waste gases such as CO₂. Batch and Fed-Batch culturing methods are common and known in the art (see, e.g., Thomas D. Brock, Biotechnology: A Textbook of Industrial Microbiology, 2^{nd} Ed. (1989) Sinauer Associates, Inc., Sunderland, MA; Deshpande, Appl. Biochem. Biotechnol. 36:227, 1992, which methods are incorporated herein by reference in their entirety).

Continuous cultures are “open” systems where a defined culture media is added continuously to a bioreactor and an equal amount of conditioned media is removed simultaneously for processing. Continuous cultures generally maintain the cells at a constant high liquid phase density where cells are primarily in logarithmic phase growth. Alternatively, continuous culture may be practiced with immobilized cells where the C₁ substrate and nutrients are continuously added and valuable products, by-products, and waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limited nutrient, such as the C₁ substrate or nitrogen level, at a fixed rate and allow all other parameters to modulate. In other systems, a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes, as well as techniques for maximizing the rate of product formation, are well known in the art.

Liquid phase bioreactors (e.g., stirred tank, packed bed, one liquid phase, two liquid phase, hollow fiber membrane) are well known in the art and may be used for growth of microorganisms and biocatalysis.

By using gas phase bioreactors, substrates for bioproduction are absorbed from a gas by microorganisms, rather than from a liquid. Use of gas phase bioreactors with microorganisms is known in the art (see, e.g., U.S. Pat. Nos. 2,793,096; 4,999,302; 5,585,266; 5,079,168; and 6,143,556; U.S. Statutory Invention Registration H1430; U.S. Pat. Appl. Pub. No. US 2003/0032170; Emerging Technologies in Hazardous Waste Management III, 1993, eds. Tedder and Pohland, pp. 411-428, all of which are incorporated herein by reference). Exemplary gas phase bioreactors include single pass system, closed loop pumping system, and fluidized bed reactor. By utilizing gas phase bioreactors, methane or other gaseous substrates are readily available for bioconversion by polypeptides with, for example, monooxygenase activity.

Suitable fermenters for culturing C₁ metabolizing non-photosynthetic bacteria (e.g., methanotrophic bacteria) may be of the loop-type or air-lift reactors. Exemplary fermenters include U-loop fermenters (see U.S. Pat. No. 7,579,163, WO2017/218978), serpentine fermenters (see WO 2018/132379), and Kylindros fermenters (see WO 2019/0366372).

In embodiments wherein the C₁ metabolizing non-photosynthetic bacterium is a methanotrophic bacterium, the methanotrophic bacteria may be grown as an isolated pure culture, with a heterologous non-methanotrophic bacterium that may aid with growth, or one or more different strains or species of methanotrophic bacteria may be combined to generate a mixed culture.

In embodiments where the C₁ metabolizing non-photosynthetic bacterium comprises Methylococcus capsulatus, the culture medium may include a biomass derived from M. capsulatus cultured with one or more heterologous organisms, such as Cupriavidus sp., Anuerinibacillus danicus or both and optionally in combination with Brevibacillus agri. In such embodiments, the bacterial biomass may comprise biomass from the heterologous organism(s) in addition to biomass from M. capsulatus.

During bacterial culture, the pH of the fermentation mixtures will generally be regulated to be between about 6 and about 8, such as between about 6 and about 7, between about 7 and about 8, or between about 6.5 and 7.5.

During bacterial culture, the temperature is maintained to be in the range optimal for the cultured bacterium. For example, for M. capsulatus Bath, the temperature may be between 40° C. and 45° C., such as 42° C.

In particular embodiments, the biomass comprises primarily (i.e., more than 50%, such as more than 55%, more than 60%, more than 65%, more than 70%, more than 75%, more than 80%, more than 85% or more than 90% by weight) biomass from M. capsulatus.

Preferably, M. capsulatus may be cultured using methane as its carbon source, air or pure oxygen for oxygenation, and ammonia as the nitrogen source. In certain embodiments, a carbon feedstock comprising methane used for culturing M. capsulatus is natural gas or unconventional natural gas. In addition to these substrates, the bacterial culture will typically require water, phosphate, and several minerals such as magnesium, calcium, potassium, irons, copper, zinc, manganese, nickel, cobalt and molybdenum. Exemplary culture media include Higgins minimal nitrate salts medium (NSM) or MM-W1 medium, master mix feed (MMF) as described in Example 1, MMF1.1, medium MMS1.0, or AMS medium. Exemplary culturing conditions of M. capsulatus are provided in the Examples.

The composition of medium MMS 1.0 is as follows: 0.8 mM MgSO₄·7H₂O, 30 mM NaNO₃, 0.14 mM CaCl₂, 1.2 mM NaHCO₃, 2.35 mM KH₂PO₄, 3.4 mM K₂HPO₄, 20.7 μM Na₂MoO₄·2H₂O, 6 μM CuSO₄·5H₂O, 10 μM Fe^{III}-Na-EDTA, and 1 mL per liter of a trace metals solution (containing per liter: 500 mg FeSO₄·7H₂O, 400 mg ZnSO₄·7H₂O, 20 mg MnCl₂·7H₂O, 50 mg CoCl₂·6H₂O, 10 mg NiCl₂·6H₂O, 15 mg H₃BO₃, 250 mg EDTA). The final pH of the media is 7.0±0.1.

The AMS medium contains the following per liter: 10 mg NH₃, 75 mg H₃PO₄·2H₂O, 380 mg MgSO₄·7H₂O, 100 mg CaCl₂·2H₂O, 200 mg K₂SO₄, 75 mg FeSO₄·7H₂O, 1.0 mg CuSO₄·5H₂O, 0.96 mg ZnSO₄·7H₂O, 120 μg CoCl₂·6H₂O, 48 μg MnCl₂·4H₂O, 36 μg H₃BO₃, 24 μg NiCl₂·6H₂O and 1.20 μg NaMoO₄·2H₂O.

The composition of medium MMF1.1 is as follows: 0.8 mM MgSO₄·7H₂O, 40 mM NaNO₃, 0.14 mM CaCl₂, 6 mM NaHCO₃, 4.7 mM KH₂PO₄, 6.8 mM K₂HPO₄, 20.7 μM Na₂MoO₄·2H₂O, 6 μM CuSO₄·5H₂O, 10 μM Fe^{III}-Na-EDTA, and 1 mL per liter of trace metals solution (containing, per liter 500 mg FeSO₄·7H₂O, 400 mg ZnSO₄·7H₂O, 20 mg MnCl₂·7H₂O, 50 mg CoCl₂·6H₂O, 10 mg NiCl₂·6H₂O, 15 mg H₃BO₃, 250 mg EDTA).

Biomass may be harvested from bacterial culture by various techniques, such as sedimentation, centrifugation, microfiltration, ultrafiltration, and spray drying. Preferably, biomass is harvested from bacterial culture by centrifugation (e.g., at 4,000×g for 10 minutes at 10° C.). For example, a fermentation broth (cells and liquid) may be collected and centrifuged. After centrifugation, the liquid can be discarded, and the precipitated cells may be saved and optionally lyophilized.

In some embodiments, the culture medium includes a derivative of the biomass. The biomass may be process by one or more additional steps to obtain a biomass derivative. As used herein the term “derivative” when used in relation to a biomass, includes any product which may be derived from such a material using a downstream processing technique or techniques known in the art, such as separation of a biomass material from a fermentation medium or liquid by centrifugation and/or filtration methods; homogenization or cell disruption by use of high pressure homogenizers or bead mills or sonication; digestion or lysis of the cells and their components by activation of endogenous enzymes or additions or external enzymes; various heat treatments; and drying by evaporation, spray drying, drum drying or freeze drying. Biomass derivatives include biomass autolysates, biomass lysates, biomass extracts, biomass isolates, biomass suspension, biomass homogenates, and biomass digestates (also referred to as “digests”). The finished media ingredient may be in the form of a flowable aqueous paste, a slurry or a dried powder.

A “biomass lysate” refers to a biomass of which cells that have been lysed (i.e., the cell wall and/or membrane of the cells have been broken down). The cell lysis may be performed for example by electrochemical lysis (e.g., using hydroxide ions that are created electrochemically within the device by a palladium electrode, porating the membrane of a cell causing cell lysis), chemical lysis (e.g., by chemically solubilizing proteins and lipids within cell membrane), acoustic lysis (e.g., using ultrasonic waves to generate high and low pressure that causes cavitation and in turn cell lysis), mechanical lysis (e.g., using physical penetration to break cell membrane).

A “biomass digestate” refers to one or more components of a biomass that have been enzymatically processed. Examples of biomass digestates include autolysates and hydrolysates, which are formed by autolysis or hydrolysis, respectively. Digestion of the biomass, such as by autolysis or hydrolysis, allows for the production of free amino acids and short-chain peptides.

A “biomass hydrolysate” refers to a biomass that has undergone digestion by enzymes exogenously supplied to the biomass.

A “biomass autolysate” refers to a biomass derivative that has undergone a digestion by enzymes naturally present in the biomass, known as autolysis. In some cases, additional exogenous enzymes (e.g. proteases, lipases, catalases) can be added to the biomass to enhance or accelerate the autolysis process. It will generally be conducted by incubation of the bacterial culture under carefully controlled conditions. Autolysis of the biomass may be performed by concentrating a culture of the C₁ metabolizing bacterium and warming the concentrated culture to a temperature of about 50-60° C., for a period of time sufficient to produce an autolysate. Following autolysis, the autolysate may be heat inactivated by at a temperature of about 70-80° C., and then a soluble fraction of the autolysate, which includes free amino acids, may be isolated. In some embodiments, an autolysate is produced by 1) fermentation of the C₁ metabolizing bacterium, (2) concentration of the fermentation product by centrifugation, filtration or evaporation, (3) homogenization, (4) autolysis with or without enzyme addition, (5) pasteurization, and (6) spray drying.

A “biomass extract” refers to a biomass component that has been separated from other components of the biomass. For example, some extracts could be enriched in heme or heme containing proteins. Other extracts could be enriched in specific recombinant proteins expressed in the C1 biomass (e.g. animal growth factors). Examples of biomass extracts that may be used for culture media include heme-enriched extracts and recombinant protein extracts.

A “biomass isolate” refers to a biomass component that has been separated and purified. For example, for some growth media applications it may be important to separate the soluble fraction from the residual particulate cell walls and cell debris, leading to a more soluble isolate and a particulate product. Examples of biomass isolates that may be used for culture media included filtrated and purified extracts, a soluble fraction or an insoluble fraction.

A “biomass suspension” refers to a mixture including biomass cells suspended in a liquid medium.

A “biomass homogenate” refers to a biomass that has been homogenized to release the contents of the cell. Homogenization of the biomass may be performed by sonication, bead homogenization, freeze/thaw cycles, with a Dounce homogenizer, or mortar and pestle. A biomass homogenate may be or include a viscous protein slurry containing both soluble and particulate cellular components.

In particular embodiments, the cell or tissue culture medium comprises a biomass of the C₁ metabolizing bacterium at a concentration of at least 0.1 g/l. In some embodiments, the amount of the biomass or derivative thereof in the culture medium is at least 0.1 g/l, at least 0.5 g/l, at least 1.0 g/l, at least 2.0 g/l, at least 3.0 g/l, at least 4.0 g/l, or at least 5.0 g/l.

In particular embodiments, the amount of the biomass or derivative thereof in the culture medium is in the range of from 0.1 to 50 g/l. In some embodiments, the amount of the biomass or derivative thereof in the culture medium is in the range of from 0.1 to 50 g/l, 0.1 to 40 g/1, 0.1 to 30 g/1, 0.1 to 20 g/1, 0.1 to 10.0 g/1, 0.1 to 5 g/1, 0.5 to 20 g/l, 0.5 to 10 g/1, 0.5 to 5 g/l, 1 to 50 g/l, 1 to 40 g/l, 1 to 30 g/1, 1 to 20 g/l, 1 to 10 g/l, 1 to 5 g/1, 5 to 50 g/1, 5 to 40 g/1, 5 to 30 g/l, 5 to 20 g/1, 5 to 10 g/1, 10 to 50 g/l, 10 to 40 g/1, 10 to 30 g/l, or 10 to 20 g/l.

In some embodiments, the biomass is an autolysate and includes one or more of the following components as a percentage of dry weight: ash at about 9-11%, nitrogen at about 10-11%, crude lipid at about 7-9%, total glucose at about 2-8%, RNA at about 3-6%, DNA at about 1-3%, total amino acids at about 50-60%, free amino acids at about 10-25%, and a-amino acids at about 3-4%.

In some embodiments, the biomass is an autolysate and includes one or more of the following components: phosphorous, sulfur, chloride, calcium, potassium, magnesium, sodium, iron, copper, and zinc, such as about 19.5 g/kg phosphorous, about 5.4 g/kg sulfur, about 7.6 g/kg chloride, about 4.7 g/kg calcium, about 8.4 g/kg potassium, about 3.0 g/kg magnesium, about 20 g/kg sodium, about 0.33 g/kg iron, about 0.9 g/kg copper, and about 0.02 g/kg zinc.

In some embodiments, the biomass is an autolysate and includes one or more of the following amino acids: aspartic acid, serine, glutamic acid, glycine, histidine, arginine, threonine, alanine, proline, tyrosine, valine, methionine, isoleucine, leucine, phenylalanine, cysteine, and tryptophan, such as in the following amounts (in g/kg): aspartic acid—about 46, serine—about 15, glutamic acid—about 72, glycine—about 34, histidine—about 12, arginine—about 32, threonine—about 23, alanine—about 59, proline—about 26, tyrosine—about 20, valine—about 40, methionine—about15, lysine—about 34, isoleucine—about 32, leucine—about 52, phenylalanine—about 28, cysteine—about 7, and tryptophan—about 11. In some embodiments, the biomass is an autolysate and includes one or more of: riboflavin and pyridoxine, such as riboflavin at about 47 mg/kg, and pyridoxine at about 55 mg/kg.

In some embodiments, the biomass is an autolysate and includes one or more of the following parameters: Crude protein between about 50% and about 70%, Total Nitrogen between 9 and 11% of dry matter; Amino Nitrogen between 1 and 4% of dry matter; Free Amino Acids from 30 to 60% Protein digestibility, in vitro, of about 80-90%; Protein solubility of about 30-85%; pH in 2% solution of about 6.5-7.5; Moisture content of about 4-12%.

The biomass or derivative thereof of the C₁ metabolizing non-photosynthetic bacterium (e.g., Methylcoccus capsulatus) may exhibit a distinct isotopic signature that allows for identification of a cell or tissue culture medium containing the C₁ metabolizing non-photosynthetic bacterium. The distinct isotopic signature of the C₁ metabolizing non-photosynthetic bacterium biomass or derivative thereof may allow for distinguishing a culture medium including the biomass or derivative thereof from a culture medium that includes a different carbon and nitrogen source such as yeast extract, peptone, or soytone. The distinct isotopic signature may include at least one, at least two, or all of: a distinct isotopic δ ¹³C value, a distinct isotopic δ ¹⁵N value, and a distinct isotopic δ ³⁴S value.

An isotopic δ ¹³C value refers to a value of a stable isotopic composition of carbon that is calculated by: (in %)=(Rₛₐₘₚₗₑ/R_{standard}−1)1000, where “R” is ¹³C:¹²C. R_{standard} for calculating an isotopic δ ¹³C value is based on the international standard Vienna Pee Dee Belmnite (VPDB).

An isotopic δ ¹⁵N value refers to a value of a stable isotopic composition of nitrogen that is calculated by: (in %)=(Rₛₐₘₚₗₑ/R_{standard}−1)1000, where “R” is ¹⁵N:¹⁴N. R_{standard} for calculating an isotopic δ ¹⁵N value is based on the atmospheric ¹⁵N:¹⁴N ratio.

An isotopic δ ³⁴S value refers to a value of a stable isotopic composition of sulfur that is calculated by: (in %)=(Rₛₐₘₚₗₑ/R_{standard}−1)1000, where “R” is ³⁴S:³²S. R_{standard} for calculating an isotopic δ ³⁴S value is based on Vienna-Canyon Diablo Troilite (VCDT).

Isotopic signatures may be measured by isotope ratio mass spectrometry. Methods of measuring isotopes are provided in, for example, Templeton et al. Geochim. Cosmochim. Acta 70:1739, 2006, which methods are hereby incorporated by reference in their entirety. In certain embodiments, the isotopic signatures are determined from a bulk sample (e.g., a complete biomass of the C₁ metabolizing non-photosynthetic bacterium) and one or more bulk reference samples (e.g., muscle of reference samples). In certain other embodiments, the isotopic signatures are determined by compound specific isotope analysis. Compound specific isotope analysis may be used to analyze an isotopic signature of, for example, a particular amino acid (e.g., glutamic acid, aspartic acid, leucine, tryptophan, tyrosine, or phenylalanine), a subset of amino acids (e.g., glutamic acid, aspartic acid, and leucine), total amino acids, total lipids or total fatty acids, saturated or unsaturated fatty acids, particular chain lengths of amino acid (e.g., C16 or C18), particular fatty acids (e.g., palmitic acid, stearic acid, palmitoleic acid), n-alkanes, or targeted hydrocarbons (e.g., isoprenoids, vitamins).

In certain embodiments, the C₁ metabolizing non-photosynthetic bacterium (e.g., Methylcoccus capsulatus) biomass or derivative thereof exhibits at least one, at least two, or all of: a δ ¹³C value that is lower than a δ ¹³C value of other carbon or nitrogen sources that are used in culture media (e.g., yeast extract, peptone, and soytone), a δ ¹⁵N value that is lower than a δ ¹⁵ value of other carbon or nitrogen sources that are used in culture media (e.g., yeast extract, peptone, and soytone), and a δ ³⁴S value that is lower than a δ ³⁴S value of other carbon or nitrogen sources that are used in culture media (e.g., yeast extract, peptone, and soytone).

In certain embodiments, the C₁ metabolizing non-photosynthetic bacterium (e.g., Methylcoccus capsulatus) of the cell or tissue culture medium, and the related biomass, exhibit a δ ¹³C of less than −30%, less than −31%, less than −32%, less than −33%, less than −34%, less than −35%, less than −36%, less than −37%, less than −38%, less than −39%, less than −40%, less than −41%, less than −42%, less than −43%, less than −44%, less than −45%, less than −46%, less than −47%, less than −48%, less than −49%, less than −50%, less than −51%, less than −52%, less than −53%, less than −54%, less than −55%, less than −56%, less than −57%, less than −58%, less than −59%, less than −60%, less than −61%, less than −62%, less than −63%, less than −64%, less than −65%, less than −66%, less than −67%, less than −68%, less than −69%, or less than −70%.

In certain embodiments, the C₁ metabolizing bacterium (e.g., Methylcoccus capsulatus) of the cell or tissue culture medium, and related biomass, exhibit a δ ¹³C of about −35to about −50%, −45% to about −35%, or about −50% to about −40%, or about −45% to about −65%, or about −60% to about −70%, or about −30% to about −70%. In further embodiments, the C₁ metabolizing non-photosynthetic bacterium is an obligate methanotroph, and the related biomass or derivative thereof exhibit a δ ¹³C value of less than about −30%, or ranges from about −40% to about −60%, or about −40% to about −50%.

The biomass or derivative thereof of the C₁ metabolizing bacterium of the culture medium may include a variety of nutrients that confer advantages to the culture medium. In particular, the nutrient profile of the biomass may provide more efficient growth of cells or tissue cultured in the culture medium, and/or may confer other desirable properties to cells or tissue cultured in the culture medium or to cell culture products derived from cells or tissue cultured in the culture medium.

In some embodiments, the biomass or derivative thereof includes heme. “Heme” refers to a porphyrin molecule that coordinates iron. “Heme iron” refers to iron coordinated by a heme molecule. Heme-iron as a dietary source of iron is more easily absorbed than non-heme iron and in a pathway that is distinct from that of non-heme-iron. Heme-iron remains soluble in the high pH environment of the upper small bowel, in contrast to inorganic, non-heme iron. As previously described, heme iron (or heme) may be linked to a protein, forming a hemoprotein.

The concentration of heme as disclosed herein is measured by a method based on the conversion of heme to the fluorescent porphyrin derivative by removal of the heme iron under acidic conditions (Sassa S (1976) Sequential induction of heme pathway enzymes during erythroid differentiation of mouse Friend leukemia virus-infected cells. The Journal of experimental medicine 143(2):305-315). The amount of heme iron is then calculated based on the 1:1 molar ratio between heme and heme iron.

In some embodiments, the biomass or derivative thereof has at least 0.01 mg, at least 0.05 mg, or at least 0.1 mg, heme per gram protein in the biomass or derivative thereof. In certain embodiments, the biomass or derivative thereof contains 0.01 to 10 mg heme/g protein, such as 0.01 to 5, 0.01 to 2, 0.01 to 1, 0.001 to 0.5, 0.05 to 10, 0.05 to 5, 0.05 to 2, 0.05 to 1, 0.005 to 0.5, 0.1 to 10, 0.1 to 5, 0.1 to 2, 0.1 to 1, 0.1 to 0.5, 0.2 to 10, 0.2 to 5, 0.2 to 2, 0.2 to 1, 0.2 to 0.5 mg heme/g protein.

In some embodiments, the biomass or derivative thereof (e.g., autolysate) includes heme iron at a concentration of at least 0.001 mg/g protein in the biomass or derivative thereof. In some embodiments, the biomass includes heme iron at a concentration of at least 0.002 mg/g, at least 0.005 mg/g, or at least 0.01 mg/g. In some embodiments, the amount of heme iron in the biomass is in a range from 0.001 to 1 mg/g, 0.005 to 1 mg/g, 0.01 to 1 mg/g, 0.001 to 0.5 mg/g, 0.005 to 0.5 mg/g, 0.01 to 0.5 mg/g, 0.001 to 0.1 mg/g, 0.005 to 0.1 mg/g, 0.01 to 0.1 mg/g, 0.001 to 0.05 mg/g, 0.005 to 0.05 mg/g, or 0.01 to 0.05 mg/g.

In some embodiments, the biomass or derivative thereof includes a desirable amount of essential amino acids. Essential amino acids refer to the amino acids that cannot be produced by the human body and include: histidine, isoleucine, leucine, lysine, methionine, phenylalanine, threonine, tryptophan, and valine. The amount of essential amino acids may refer to the total amount of all nine essential amino acids. Amino acid content of a sample may be measured by LC-mass spectrometry or high performance liquid chromatography. Total essential amino acids may be calculated by measuring the sum total weight of each of histidine, isoleucine, leucine, lysine, methionine, phenylalanine, threonine, tryptophan, and valine present in a sample. In some embodiments, the biomass includes essential amino acids at an amount of at least 1 mg/g. In some embodiments, the biomass includes essential amino acids at an amount of at least 1 mg/g, at least 2 mg/g, at least 5 mg/g, or at least 10 mg/g. In some embodiments, the biomass includes essential amino acids at an amount of at least 1 mg/g. In some embodiments, the biomass includes essential amino acids at an amount within a range of 1 to 100 mg/g, 2 to 100 mg/g, 5 to 100 mg/g, or 10 to 60 mg/g.

In some embodiments, the biomass or derivative thereof includes a desirable amount of at least one essential amino acid. Essential amino acids as used herein include valine, leucine, isoleucine, phenylalanine, methionine, lysine, threonine, histidine, and tryptophan. In some embodiments, the biomass or derivative thereof comprises essential amino acids at an amount within the range of 1-100 mg/g each, such as 10-80 mg/g each, 20-60 mg/g each. In some embodiments, the biomass includes 1-100 mg/g, such as 10-80 mg/g and 20-60 mg/g, or at least 1 mg/g, at least 2 mg/g, or at least 5 mg/g, or at least 10 mg/g of at least one essential amino acid. In some embodiments, the biomass includes 1-100 mg/g, such as 10-80 mg/g and 20-60 mg/g, or at least 5 mg/g, at least 10 mg/g, at least 15 mg/g, or at least 20 mg/g of lysine. In some embodiments, the biomass includes 1-100 mg/g, such as 10-80 mg/g and 20-60 mg/g, or at least 1 mg/g, at least 2 mg/g, at least 5 mg/g, at least 10 mg/g or at least 15 mg/g of methionine. In some embodiments, the biomass includes 1-100 mg/g, such as 10-80 mg/g and 20-60 mg/g, or at least 5 mg/g, at least 10 mg/g, at least 15 mg/g, or at least 20 mg/g of valine. In some embodiments, the biomass includes 1-100 mg/g, such as 10-80 mg/g and 20-60 mg/g, or at least 5 mg/g, at least 10 mg/g, at least 15 mg/g, or at least 20 mg/g of leucine. In some embodiments, the biomass includes 1-100 mg/g, such as 10-80 mg/g and 20-60 mg/g, or at least 5 mg/g, at least 10 mg/g, at least 15 mg/g, or at least 20 mg/g of isoleucine. In some embodiments, the biomass includes 1-100 mg/g, such as 10-80 mg/g and 20-60 mg/g, or at least 5 mg/g, at least 10 mg/g, at least 15 mg/g, or at least 20 mg/g of phenylalanine. In some embodiments, the biomass includes 1-100 mg/g, such as 10-80 mg/g and 20-60 mg/g, or at least 5 mg/g, at least 10 mg/g, at least 15 mg/g, or at least 20 mg/g of threonine. In some embodiments, the biomass includes 1-100 mg/g, such as 10-80 mg/g and 20-60 mg/g, or at least 5 mg/g, at least 10 mg/g, at least 15 mg/g, or at least 20 mg/g of tryptophan. In some embodiments, the biomass includes 1-100 mg/g, such as 10-80 mg/g and 20-60 mg/g, or at least 5 mg/g, at least 10 mg/g, at least 15 mg/g, or at least 20 mg/g of histidine.

In some embodiments, the biomass or derivative thereof includes total amino acids at a concentration of at least 20% by weight, at least 30% by weight, or at least 40% by weight. In some embodiments, the biomass includes free amino acids at a concentration of at least 1% by weight, at least 5% by weight, at least 10% by weight, or at least 15% by weight. Total amino acids may be measured by LC-mass spectrometry or high performance liquid chromatography.

In some embodiments, the biomass or derivative thereof includes copper, preferably bioavailable copper. Copper is an essential mineral that is naturally present in some and is a cofactor for several enzymes (known as “cuproenzymes”) involved in energy production, iron metabolism, neuropeptide activation, connective tissue synthesis, and neurotransmitter synthesis. “Bioavailable copper” refers to forms of copper that are readily absorbed by the body. Bioavailability of copper is affected by multiple factors. For example, plant-derived copper is less bioavailable than other dietary copper sources due to the presence of phytates and fiber. In some embodiments, the biomass or derivative thereof comprises copper at an amount within the range of 50-500 mg/kg. In some embodiments, the amount of copper in the biomass is at least 50 mg/kg, at least 75 mg/kg, or at least 100 mg/kg. In some embodiments, the amount of bioavailable copper in the biomass is in the range of from 50 to 350 mg/kg, 75 mg/kg, or 100 mg/kg. Copper may be measured, for example, by stable isotope measurement of ⁶⁵Cu using thermal ionization and magnetic sector mass spectrometry (see e.g., Turnlund, J., Science of The Total Environment (28), 1-3, 1983, 385-392).

In particular embodiments, the culture medium comprises one or more further ingredients in addition to the C₁ metabolizing bacterium. The further ingredients may be chosen based on the type of cells or tissue that will be cultured in the culture medium. Further ingredients of the culture medium may include one or more of: a liquid or non-liquid carrier or diluent (e.g., water, a gel such as an agar gel, a gellable liquid); mineral salts; carbohydrates such as saccharides, organic alcohols (e.g., glycerol), and other carbon sources including organic acids (e.g., lactic acid or lactate); nitrogen sources such as nitrates, protein fragments, ammonium compounds, amino acids and particularly essential amino acids such as tryptophan; nucleic acids or nucleic acid fragments; and lipids. In certain embodiments, the further ingredients include a saccharide such as glucose or dextrose. In certain embodiments, the further ingredients include a mineral salt such as potassium, calcium, magnesium, sodium, molybdenum, iron, zinc, boron, cobalt, manganese, or nickel. In certain embodiments, the further ingredients include a complex component such as a crude agricultural product such as corn steep liquor, a yeast extract or peptone.

The culture medium may be a liquid medium or a solid medium, depending on the cell type to be cultured. In particular embodiments, the culture medium is a liquid medium. In particular embodiments, the culture medium is a solid medium. Solid medium may be produced, for example, by mixing a liquid medium with a gelling agent such as agarose, and allowing the medium to cool and solidify.

In some embodiments, the biomass or derivative thereof of the hemoprotein-producing C₁ metabolizing non-photosynthetic bacterium is used as a primary nitrogen source in culture medium. The biomass or derivative thereof may be used to replace a primary nitrogen source and optionally one or more other nitrogen sources used in culture media.

Primary nitrogen sources used in culture media include animal-free extracts and animal-based extracts. Animal-free extracts used in culture media include yeast extracts, soy extracts, malt extracts, vegetal peptone, and microbial peptone. Animal-free extracts may have a nitrogen content of approximately 10%. Animal-based extracts used in culture media include and meat extracts include beef extracts (e.g., Beef Extract Powder, BBL™ and Bacto™ Tryptose), porcine extracts (e.g., Proteose Peptone No. 3), casein and wheat extracts (e.g., BBL™ Trypticase, TC Lactalbumin, Acidicase Peptone BBL, Biosate Peptone BBL, Casamino Acids, Bacto, Casein digest Difco, Casitone, Bacto). Animal based extracts used in cell culture may have a nitrogen content that is higher than that of animal-free extracts (e.g., approximately 13%).

In some embodiments, the biomass or derivative thereof replaces a peptone as a primary nitrogen source in the culture medium. Peptone is an organic compound providing carbon source, organic nitrogen source, growth factors and other nutrients for the microorganisms, cell. Peptone is obtained from meat, casein, gelatin, soy, pea, wheat, potato, and other proteins. The major types of peptone include animal peptone, vegetal peptone, and microbial peptone. Peptone is a water-soluble complex derived from hydrolysis during the protein digestion process. It is an organic compound and a source of inorganic nitrogen, peptides, and proteins in the growth of the microorganisms and cells. Peptones are obtained by partially break down of proteins either by acid hydrolysis or by enzymes into short peptides and amino acids. Peptone composition depends on the source of the protein and digestion process these factors determine the relative prevalence of amino acids and peptides.

Examples of peptones include soy-based peptones (e.g., Phytone™ Peptone, Select Soytone), porcine-based peptones (e.g., Proteose Peptone No. 3), milk-based peptones (e.g., TC Lactalbumin), meat-based peptones (e.g., Bacto™ Tryptose, BBL Beef Extract Powder, Galysate Peptone, Neopeptone, Bacto Peptone, Polypeptone Peptone, Procose Peptone, Thiotone), and yeast-based peptones (e.g., TC Yeastolate, Bacto™ Yeast Extract).

In some embodiments, the biomass or derivative thereof replaces a yeast extract as a primary nitrogen source in the culture medium. There are two different types of yeast extracts: hyrolyzed yeast extract, also called yeast peptone, and autolyzed yeast. The hyrolyzed yeast extract is produced by digestion of exogeneous enzymes or acid to hydrolyze the proteins. A yeast autolysate or yeast autolysate extract is made by fermentation of yeast to a concentration level where the yeast dies and the cells walls break. The proteases from the yeast itself start the digestion of the proteins and split them into peptides and amino acids. The insoluble portion is removed.

In some embodiments, the culture medium is suitable for bacterial cell culture. Cell culture media that are commonly used for bacterial cell culture include tryptic soy broth (TSB); lysogeny broth (LB, also known as Luria-Bertani broth); media selective for gram negative bacteria such as Hektoen enteric agar, MacConkey agar, and xylose lysine deoxycholate; and media selective for gram positive bacteria such as mannitol salt agar. The culture medium disclosed herein that is suitable for bacterial culture may comprise one or more ingredients of such known culture media.

In particular embodiments, the culture medium is suitable for marine-dwelling bacteria. Culture medium suitable for marine-dwelling bacteria may include filtered seawater. An example of culture medium that is suitable for marine-dwelling bacteria is Difco™ Marine Broth 2216. The culture medium disclosed herein that is suitable for marine-dwelling bacterial culture may comprise one or more ingredients of known marine-dwelling bacterial culture media.

In some embodiments, the culture medium is suitable for a Bacillus species such as B. subtilis and B. licheniformis. In some embodiments, the culture medium suitable for a Bacillus species (e.g., B. subtilis and B. licheniformis) includes at least one of KCl, MgCl₂, NaCl, and CaCl₂ at concentrations of, for example, about 0.75, about 2.5, about 0.5, and about 5.0 g/L, respectively. In some embodiments, the culture medium suitable for a Bacillus species includes glucose (e.g., at about 20 g/L), beef extract (e.g., at about 9 g/L), KCl (e.g., at about 0.75 g/L), and NaCl (e.g., 0.5 g/L). In some embodiments, the culture medium suitable for a Bacillus species includes aqueous filtered solutions of potatoes (Solanum tuberosum), soya chunks (Glycine max) and/or Chickpeas (Cicer arietinum).

In some embodiments, the culture medium is suitable for Escherichia coli. Examples of known culture media suitable for E. coli include LB Broth and LB Agar, and M9 minimal broth. The culture medium disclosed herein that is suitable for E. coli culture may comprise one or more ingredients of such known culture media.

In some embodiments, the culture medium is suitable for Corynebacterium glutamicum. An example of known culture medium suitable for C. glutamicum is CGXII, which may optionally be supplemented with brain-heart-infusion (BHI) and/or amino acid (AA) cocktails. The culture medium disclosed herein that is suitable for C. glutamicum culture may comprise one or more ingredients of such known culture media.

In some embodiments, the culture medium is suitable for Pseudomonas putida. Examples of known known culture media suitable for P. putida include LB Broth, LB Agar, and EWING's media. The culture medium disclosed herein that is suitable for P. putida culture may comprise one or more ingredients of such known culture media.

In some embodiments, the culture medium is suitable for a Xanthomonas species. Examples of known culture media suitable for a Xanthamonas species are peptone sucrose agar (PSA), nutrient broth yeast extract medium (NBY), growth factor (GF) agar, and modified Wakimoto's agar. The culture medium disclosed herein that is suitable for a Xanthomonas species culture may comprise one or more ingredients of such known culture media.

In some embodiments, the culture medium is suitable for non-bacterial cell culture. A culture medium suitable for non-bacterial cell culture may include an anti-bacterial agent. Anti-bacterial agents are agents such as small molecules that inhibit the growth of and/or or kill bacterial organisms. Examples of anti-bacterial agents include kanamycin, streptomycin, and penicillin.

In some embodiments, the culture medium is suitable for algal cell culture. Algal cell culture medium may include seawater base and/or soil extract. Seawater base refers to natural seawater (for example, sterile-filtered seawater) or synthetic seawater that includes purified water and set amounts of salts mimicking the content of seawater. Soil extract may be produced by producing sterile-filtering a suspension of soil in water. Examples of algal cell culture media include soilwater medium, waris medium and Guillard's F/2 medium. The culture medium disclosed herein that is suitable for algal culture may comprise one or more ingredients of known algal culture media.

In some embodiments, the culture medium is suitable for fungal cell or tissue culture. A fungal cell or tissue culture medium may include an anti-bacterial agent. Examples of culture media commonly used for fungal cell or tissue culture include YPD broth, CSM media, Yeast Nitrogen Base, and potato dextrose broth. The culture medium disclosed herein that is suitable for fungal cell or tissue culture may comprise one or more ingredients of such commonly used broths, such as glucose, dextrose, yeast extract, potato extract, and peptone.

In some embodiments, the culture medium is suitable for yeast cell culture. Examples of culture media commonly used for yeast cell culture include YPD broth, CSM media, and Yeast Nitrogen Base. The culture medium disclosed herein that is suitable for yeast cell culture may comprise one or more ingredients of such commonly used broth, such as glucose, dextrose, yeast extract, and peptone.

In some embodiments, the culture medium is suitable for mushroom cell or tissue culture. Culture medium suitable for mushroom cell or tissue culture may include potato extract, grain, and/or fruiting substrate. Potato extract can be made by boiling washed but unpeeled potatoes in distilled water and then decanting or straining the broth through cheesecloth. Fruiting substrate refers to a substrate for growth of mycelium, which may include one or more of: straw, dead logs, sawdust, woodchips, grain such as wheat bran, and coffee grounds. Examples of media useful for growth of mycelium include potato dextrose broth or potato dextrose agar, yeast extract broth or agar, malt extract or agar, lamberts agar and compost extract broth or agar, cornmeal extract, and oat extract. Additionally, the following are examples of media and recipes for producing one liter of the media:
PDA—Potato Dextrose Agar Medium: Potato dextrose agar—39 g, Water—1000 ml; MEA—Malt Extract Agar Medium:
Malt extract −30 g, Agar-Agar—15 g, Water—1000 ml;
GPA—Glucose Peptone Agar Medium: Peptone—20 g, Dextrose—10 g, Nacl—5 g, Agar-Agar—15 g, Water—1000 ml;
YMA—Yeast Malt Agar Medium: Malt—20 g, Yeast—2 g, Agar-Agar—15 g, Water—1000 ml; and
SDA—Saboraud's Dextrose Agar Medium: Dextrose 40 g, Agar-Agar—15 g, Peptone—10 g, Water—1000 ml.

An example of culture media suitable for mushroom cell or tissue culture is potato dextrose broth. Potato dextrose broth can be made by boiling 200 grams of washed but unpeeled potatoes in 1 liter of distilled water for 30 minutes and then decanting or straining the broth through cheesecloth. Distilled water is added such that the total volume of the suspension is 1 liter. 20 grams of dextrose is then added and the medium is sterilized by autoclaving.

The culture medium disclosed herein that is suitable for mushroom cell or tissue culture may comprise one or more of the ingredients of known mushroom culture media.

In some embodiments, the culture medium is suitable for animal cell or tissue culture. The culture medium may comprise one or more ingredients of known animal cell or tissue culture media. A typical culture medium for animal cells is composed of a complement of amino acids, vitamins, inorganic salts, glucose, and serum as a source of growth factors, hormones, and attachment factors. Culture medium suitable for animal cell or tissue culture may include serum, a growth hormone, a growth factor, an antibacterial agent and/or an antifungal agent. Culture media used for animal cells or tissue may include a media base such as Modified Essential Media (MEM), Dulbecco's Modified Eagle's Medium (DMEM), RPMI-1640 (“RPMI), Eagle's Minimum Essential Medium (EMEM), Iscove's Modified Dulbecco's Medium (IMDM), or Ham's F12. Culture media used for animal cells may additionally include a balanced salt solution such as phosphate-buffered saline, Dulbecco's phosphate-buffered saline, and Hanks' Balanced Salt Solution.

In some embodiments, the culture medium suitable for animal cells or tissue includes L-glutamine. L-glutamine is an essential amino acid. “Essential amino acid” refers to an amino acid that a cell type cannot produce itself. L-glutamine provides nitrogen for NAD, NADPH and nucleotides and serves as a secondary energy source for metabolism. L-glutamine is an unstable amino acid that, with time, converts to a form that cannot be used by cells, and should thus be added to media just before use.

In some embodiments, the culture medium includes serum. Serum may be added to the culture medium as a source of growth factors, hormones, and attachment factors. Additionally, serum provides carriers or chelators for labile or water-insoluble nutrients, protease inhibitors, and also binds and neutralizes toxic moieties. Serum is commonly added to animal cell culture medium at a concentration of 2-10%. Examples of serum that may be used include bovine serum such as fetal bovine serum, chicken serum, horse serum, human serum, and fish serum. Fetal bovine serum is the serum most commonly added to culture media.

In some embodiments, the culture medium does not include serum. Reasons it may be beneficial to exclude serum include: batch inconsistency, potential for contaminating cultures, animal welfare concerns, and supply issues. In some embodiments, it is particularly useful to exclude serum from media used for producing food ingredient. In some embodiments, use of the C₁ metabolizing microorganism biomass or derivative thereof in the medium allows for the exclusion of serum without sacrificing growth efficiency of the cells cultured in the medium. In some embodiments, the medium includes a serum substitute, such as Ultroser G (particularly useful for growth of eukaryotes) or a mushroom extract. In some embodiments, the C₁ metabolizing microorganism biomass or derivative thereof serves as a serum substitute.

In some embodiments, the culture medium includes growth factors. Growth factors are naturally occurring substances capable of stimulating cell proliferation, wound healing, and occasionally cellular differentiation. Growth factors may be supplied to media through serum, or may be added to the media independent from serum. Examples of growth factors include fibroblast growth factor, erythropoietin, ephrin, hepatocyte growth factor, and insulin-like growth factor. Growth hormone refers to a peptide hormone that stimulates growth, such as human growth hormone (hGH).

In certain embodiments, culture media suitable for animal cells or tissue disclosed herein comprises one or more anti-fungal agents. Anti-fungal agents are agents such as small molecules that inhibit the growth of and/or or kill fungal cells. Examples of anti-fungal agents include amphotericin B, voriconazole, and caspofungin.

Examples of culture media used for animal cells and tissue include MEM+2 mM Glutamine+10% FBS +1% Non Essential Amino Acids (NEAA); RPMI 1640+2 mM Glutamine+10-20% FBS; DMEM+2 mM Glutamine+5% New Born Calf Serum (NBCS)+5% FBS; DMEM+2 mM Glutamine+10% FBS; Ham's F12+2 mM Glutamine+10% FBS; EMEM (EBSS)+2 mM Glutamine+1% Non Essential Amino Acids (NEAA)+10% FBS; F-12 K+10% FBS+100 μg/ml Heparin; and RPMI-1640+10% FBS.

In some embodiments, the culture medium is suitable for fish or shellfish cell or tissue culture. Eagle's MEM is an example of a cell culture medium that can be used for fish cell or tissue culture (Fernandez et al. Gyobyo Kenkyu, 28 (1), 27-34, 1993). Grace's medium, Leibovitz's-15 (L-15) medium, and M199 medium are examples of culture medium that may be used for invertebrate cells, such as shellfish cells. Examples of ingredients commonly used in fish tissue culture include serum, long-chain omega-3 fatty acids such as EPA (cicosapentaenoic acid) and DHA (docosahexaenoic acid) and other fatty acids, vitamin E, and growth factors such as fibroblast growth factor. Examples of ingredients that may be used in shellfish culture include serum and hemolymph extracts, such as derived from Penaeus species (i.e., prawns). Hemolymph refers to a fluid analogous to blood in vertebrates, which circulates in the interior of an arthropod body, while remaining in contact with the animal's tissues. The culture medium disclosed herein that is suitable for fish cell or tissue culture may comprise one or more of the ingredients of known culture media for fish or shellfish cell or tissue culture, such as fish serum. Fish Serum is a cell culture grade serum that may be derived from aseptically-drawn whole blood and plasma products from salmonid fish.

In some embodiments, the culture medium is suitable for avian cell or tissue culture. Examples of culture media that can be used to culture avian cells or tissue include Minimum Essential Medium (MEM) and Dulbecco's Modified Eagle's Medium (DMEM). In some embodiments, the medium includes serum such as fetal bovine serum, or a serum substitute. The culture medium disclosed herein that is suitable for avian cell or tissue culture may comprise one or more of the ingredients of known culture media for avian cell or tissue culture, such as glucose and chicken serum.

In some embodiments, the culture medium is suitable for insect cell or tissue culture. Examples of culture media that can be used for insect cell or tissue culture include ExpiSf CD Medium, Sf-900 III SFM, and Sf-900 II SFM, and a medium including IPL-41 basal medium, soy protein hydrolysate, yeastolate, lipid-sterol emulsion, and Pluronic F-68 (Donaldson, M. S., and Shuler, M. L. (1998). Biotechnol. Prog. 14, 573-579). The culture medium disclosed herein that is suitable for insect cell or tissue culture may comprise one or more of the ingredients of known culture media for insect cell or tissue culture, such as soy extract, yeast extract, glucose, and lactalbumin.

In some embodiments, the biomass or derivative thereof of the C₁ metabolizing bacterium is provided in the cell culture medium as a replacement for a common primary nutrient source (e.g., nitrogen source or carbon source), such as a yeast extract or yeast peptone, a soy peptone, a casein or whey peptone, or a meat peptone. A “primary” nutrient source refers to a nutrient source that provides more than 50% a particular nutrient source. For example, a primary nitrogen source refers to a nitrogen source that provides more than 50% nitrogen in a culture medium. In some embodiments, the biomass or derivative thereof is the sole or primary source of a nutrient such as nitrogen or carbon. For example, for a culture medium that includes Bacto-tryptone, yeast extract, and sodium chloride (e.g., Luria-Bertani broth), one or both of the Bacto tryptone and the yeast extract may be replaced with the biomass or derivative thereof of the C₁ metabolizing bacterium.

In particular embodiments, a cell culture medium that normally includes a common nutrient source, may be modified to replace the common nutrient source with the biomass of the C₁ metabolizing bacterium, at a substitution ratio range of about 1:10 to about 10:1, about 1:5 to about 5:1, or about 1:2 to about 2:1 (common source: biomass of the C₁ metabolizing bacterium, by weight). In particular embodiments, a cell culture medium that normally includes a common nutrient source may be modified to replace the common nutrient source with the biomass of the C₁ metabolizing bacterium, at a substitution ratio of about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, or about 10:1 (common source: biomass of the C₁ metabolizing bacterium, by weight).

The present disclosure also provides concentrates of cell or tissue culture media described herein. Such concentrates may be diluted into cell or tissue culture media suitable for culturing various types of cells or tissue (e.g., suitable for culturing bacterial cells or suitable for culturing non-bacterial cells or tissue). The concentrates may be in a liquid, semi-solid (e.g., gel), or solid state.

### B. Methods of Culturing Cells or Tissue

As previously described, methods cell or tissue culture or fermentation are provided herein.

In some embodiments, the method comprises culturing cells or tissue in a culture or fermentation medium that comprises a biomass or derivative thereof of a hemoprotein-producing C₁ metabolizing non-photosynthetic bacterium provided herein. In certain embodiments, the amount of heme in the biomass or derivative thereof is in the range of from 0.01 to 10.0 mg/g protein, and/or the amount of the biomass or derivative thereof in the culture medium is in the range of 0.1 to 20 g/l.

In some embodiments, the methods include culturing bacterial cells. In some embodiments, the bacterial cells are selected from Bacillus species such as B. subtilis and B. licheniformis, Escherichia coli, Corynebacterium glutamicum, Pseudomonas putida, Xanthomonas species such as Xanthomonas campestris, marine dwelling bacteria and phytoprotective bacteria. In some embodiments, the bacterial cells are bacteria used for food or beverage fermentation, and/or are used as a probiotic. Examples of microorganisms used in probiotics include several Lactobacillus species (e.g., Lb. fermentum, Lb. acidophilus, Lb. rhamnosus, and Lb. reuteri), Bifidobacterium lactis, Bf. bifidum, Bf. longum, Bf. infantis, Bf. animalis, Bf. breve, Saccharomyces boulardii, Streptococcus thermophilus, and Bacillus coagulans. “Probiotic” may refer to a live microorganism culture that is administered in a live form and confers health advantages. Orally administered probiotics are capable of passing through the gastrointestinal tract in a live form.

In some embodiments, the bacteria are marine dwelling bacteria. Examples of marine dwelling bacteria that may be cultured include Shewanella species such as S. pneumatophori, Photobacterium profundum, Moritella marina, and Vibrio species. Shewanella species and Moritella marina are marine dwelling bacteria that are capable of producing omega-3 fatty acids such as docosahexaenoic acid (DHA) and eicosapentaenoic acid (EPA).

In particular embodiments, the bacteria are phytoprotective bacteria. “Phytoprotective bacteria” are bacteria capable of protecting plants from pathogens, such as bacterial pathogens, pathogenic nematodes, and/or pathogenic fungi. Examples of phytoprotective bacteria include Bacillus methylotropicus and B. subtilis, B. licheniformis, and B. amyloliquefaciens, Bacillus thuringiensis.

In some embodiments, the bacteria are used for food or beverage fermentation. Examples of bacteria used for food or beverage fermentation include Lactococcus species, Lactobacillus species, Streptococcus species, Bifidobacterium species, Pediococcus species, Micrococcus species, Leuconostoc species, Staphylococcus species, and Penicillium nalgiovense.

In some embodiments, the methods include culturing non-bacterial cells or tissue. The non-bacterial cells or tissue may include algal cells, fungal cells, and/or animal cells.

In some embodiments, the methods include culturing algal cells. Algae are predominantly aquatic photosynthetic organism that include microalgae (referring to unicellular algae) and macroalgae (referring to multicellular algae). In particular embodiments, the algal cells are Schizochytrium, which is a marine microalgae that is capable of producing omega-3 fatty acids such as docosahexaenoic acid (DHA) and eicosapentaenoic acid (EPA).

In some embodiments, the methods include culturing fungal cells or tissue. Fungal cells or tissue includes yeast cells and mushroom cells or tissue. Examples of fungi or yeasts used in the production of ingredients of interest are Aspergillus niger, Trichoderma, Aspergillus oryzae, Ashbya gossypii, Morteriella isabellina and Mucor circinelloides.

In some embodiments, the methods include culturing yeast cells. In particular embodiments, the yeast cells are Saccharomyces cerevisiae or Brettanomyces, such as B. bruxellensis, Pichia pastoris or B. claussenii.

In some embodiments, the methods include culturing mushroom cells or tissue. To cultivate mushrooms, a mushroom spawn may be started from spores or mushroom tissue. The spawn may be cultured in a media, such as an agar media that includes a biomass of the hemoprotein-producing C₁ metabolizing non-photosynthetic bacterium. Next, the spawn may be seeded on a substrate such as a log, to produce fruiting bodies. Examples of mushrooms that may be used include shiitake (Lentinula edodes), oyster mushrooms (Pleurotus spp.), and white button mushroom (Agaricus brunnescens).

In some embodiments, the methods include culturing animal cells or tissue. The animal cells may be cultured in a liquid culture suspension such as in a flask or in one or more layers in a dish or plate, depending on the cell type. Examples of animal cells that may be used include fish or shellfish cells or tissue, insect cells or tissue, avian cells or tissue, or mammalian cells or tissue.

Culturing animal cells may be useful for producing cell-based meat products. “Cell-based meat product” refers to meat produced by in vitro culture of animal cells or tissue, instead of from slaughtered animals. Cells useful for producing cell-based meat products include embryonic stem cells, adult stem cells, myosatellite cells, myoblasts, myocytes, and/or muscle cells. In some embodiments, the animal cells comprise embryonic stem cells, adult stem cells, myosatellite cells, myoblasts, myocytes, and/or muscle cells. Cell-based meat products produced by the methods disclosed herein may have improved flavor (e.g., more metallic or more umami flavor) and/or visual appeal (e.g., redder color) as compared to cell-based meat products produced by culturing the animal cells or tissue in the absence of a hemoprotein-producing C₁ metabolizing non-photosynthetic bacterium.

In some embodiments, the methods include culturing fish or shellfish cells or tissue. Fish or shellfish cells or tissue may be cultured to produce cell-based seafood. “Cell-based seafood product” refers to an edible fish or shellfish product that is produced by in vitro culture of fish or shellfish cells or tissue, instead of from whole animals. In some embodiments, the fish or shellfish cells or tissue comprise fish muscle tissue. Examples of shellfish cells or tissue include crustacean and mollusk cells and tissue. The cell-based seafood products produced by the methods disclosed herein may have an improved “fishy” or umani flavor as compared to culturing the fish or shellfish cells in the absence of a hemoprotein-producing C₁ metabolizing non-photosynthetic bacterium.

In some embodiments, the methods include culturing avian cells or tissue. Avian cells or tissue may be used to produce cell-based poultry products. “Cell-based poultry product” refers to a poultry product that is produced by in vitro culture of avian cells or tissue, instead of from a whole animal. The avian cells may include chicken cells, turkey cells, quail cells, duck cells, goose cells. For example, chicken muscle cells or tissue may be grown to produce cell-based chicken products.

In some embodiments, the methods include culturing insect cells or tissue. Insect cells, such as insect muscle cells or fat body cells, may be cultured as a food source. Insect cells that are commonly grown in cell culture include Bombyx mori, Mamestra brassicae, Spodoptera frugiperda, Trichoplusia ni, and Drosophila melanogaster.

In some embodiments, the methods include culturing mammalian cells or tissue. Mammalian cells or tissue may be useful for producing cell-based meat products. Examples of mammalian cells include porcine (e.g., pig or boar) cells, bovine (e.g., cow or bison) cells, sheep cells, goat cells, kangaroo cells, and guinea pig cells. For example, porcine muscle cells or tissue may be used to produce cell-based pork products.

In some embodiments, the methods include separating the cultured cells from the growth medium to produce isolated culture cells and/or an isolated supernatant. In some embodiments, the cells are separated from the supernatant by centrifugation and/or filtration.

In some embodiments, the methods include isolating a desired product from the cultured cells or tissue. The desired product may be selected from vitamins, fatty acids, amino acids, nucleosides, peptides, proteins, enzymes, pigments, flavors, fragrances, organic acids, preservatives, small molecule metabolites, ferment, culture, probiotics, and cell-based meats. A small molecule metabolite is a low molecular weight (e.g., up to 1500 daltons) organic compound, typically involved in a biological process as a substrate or product. Examples of small molecule metabolites include acetic acid, citric acid, lactic acid, isoascorbic acid, and glycerol. In some embodiments, the desired product is a biomass, or a derivative thereof, of the cells or tissue that were cultured in the culture media.

Culturing cells or tissue in a culture medium including a biomass or derivative thereof of a C₁ metabolizing bacterium as described herein may result in faster or more efficient growth of the cells or tissue. “Faster or more efficient growth” refers to an ability of the cells to grow or divide at a faster rate. In some embodiments the cells or tissue cultured in a culture medium including the biomass or derivative thereof of the C₁ metabolizing bacterium grow at a rate that is at least 5% faster, at least 6% faster, at least 7% faster, at least 8% faster, at least 9% faster, at least 10% faster, at least 15% faster, at least 20% faster, at least 25% faster, at least 30% faster, at least 35% faster, at least 40% faster, at least 45% faster, or at least 50% faster growth, as compared to equivalent cells or tissue cultured in the absence of the biomass or derivative thereof. Growth efficiency may be measured by counting cells such as by microscopy or by spectrophotometry, and plotting the cell numbers over a time-course to obtain a growth rate. The faster or more efficient growth may be based on the high nutrient profile of the biomass. In some embodiments, culturing cells or tissue by the methods described herein results in a faster growth rate of the cells or tissue, as compared to culturing the cells or tissue in a reference cell or tissue culture medium.

In some embodiments, the cells or tissue cultured in a culture medium including the biomass or derivative thereof of the C₁ metabolizing bacterium provide an enhanced yield of a desired product produced by (i.e., an enhanced productivity of) the cultured cells or tissue, wherein the enhanced yield is at least 2% greater, 5% greater, at least 6% greater, at least 7% greater, at least 8% greater, at least 9% greater, at least 10% greater, at least 15% greater, at least 20% greater, at least 25% greater, at least 30% greater, at least 35% greater, at least 40% greater, at least 45% greater, or at least 50% greater (as measured by weight), as compared to a desired product produced by equivalent cells or tissue cultured in the absence of the biomass or derivative thereof.

In some embodiments, the cells or tissue cultured in a culture medium including the biomass or derivative thereof of the C₁ metabolizing bacterium provide an enhanced yield of the cells or tissue during culturing, wherein the enhanced yield is at least 2% greater, 5% greater, at least 6% greater, at least 7% greater, at least 8% greater, at least 9% greater, at least 10% greater, at least 15% greater, at least 20% greater, at least 25% greater, at least 30% greater, at least 35% greater, at least 40% greater, at least 45% greater, or at least 50% greater (as measured by weight), as compared to the yield of equivalent cells or tissue cultured in the absence of the biomass or derivative thereof.

In some embodiments the cells or tissue cultured in a culture medium including the biomass or derivative thereof of the C₁ metabolizing bacterium provide an enhanced efficiency (i.e., an enhanced rate) of producing a product of interest by the culture cells or tissue, wherein the enhanced efficiency is at least 2% greater, 5% greater, at least 6% greater, at least 7% greater, at least 8% greater, at least 9% greater, at least 10% greater, at least 15% greater, at least 20% greater, at least 25% greater, at least 30% greater, at least 35% greater, at least 40% greater, at least 45% greater, or at least 50% greater (as measured by weight), as compared to the efficiency of producing the product of interest by equivalent cells or tissue cultured in the absence of the biomass or derivative thereof.

A “reference culture medium” as used herein refers to a culture medium that is identical to the culture medium including a biomass or derivative thereof of a hemoprotein-producing C₁ metabolizing non-photosynthetic bacterium, except that the reference culture medium includes a primary nitrogen source (e.g., yeast extract, a peptone, another primary nitrogen source described herein or used in known cell or tissue culture media) that is not derived from a hemoprotein-producing C₁ metabolizing non-photosynthetic bacterium. A reference culture medium does not comprise any biomass or derivative thereof of a hemoprotein-producing C₁ metabolizing non-photosynthetic bacterium.

### C. Cell or Tissue Culture or Fermentation Products

As previously described, cell or tissue culture or fermentation products are provided herein. The cell or tissue products may be produced by the methods of culturing cells or tissue as previously described. The method of culturing cells or tissue may include additional steps beyond culturing the cells or tissue, such as separating the cultured cells from the growth medium to produce isolated culture cells and/or an isolated supernatant, and/or isolating a desired product from the cultured cells or tissue. In some embodiments, processing the cell or tissue culture product includes isolating, concentrating, separating and/or purifying a desired product from the fermented or cultured cells or tissue.

In some embodiments, the cell or tissue culture or fermentation product includes isolated culture cells. In some embodiments, the isolated culture cells are live isolated culture cells. Examples of cell culture product that include live isolated culture cells may include probiotics, dairy cultures, meat-curing culture, phytoprotective bacterial cell products, and yeast starter cultures for baking or alcoholic beverage production. “Dairy culture” refers to a live microorganism culture, typically bacterial culture, which is added to a milk product to produce a fermented dairy product such as cheese, yogurt, buttermilk, and sour cream or kefir. Examples of dairy cultures include Lactobacillus and Bifidobacterium. “Meat-curing culture” refers a live microorganism culture that is added to meats to produced fermented or cured meat products such as sausages.

In some embodiments, the cell or tissue culture or fermentation product includes an isolated supernatant. Examples of products that would be an isolated supernatant include: specific amino acids (e.g., lysine and threonine), peptides and proteins, fatty acids (e.g. EPA and DHA), organic acids (e.g. citric acid), enzymes (e.g., chymosin, protease, lipase, amylase, cellulase, and carbohydrase), pigments (e.g., carotenoids), flavors and fragrances (e.g., vanillin and menthol), ferment, culture, probiotics (e.g., Bifidobacterium bifidum, Streptococcus thermophilus, and Bacillus thuringiensis), Vitamins (e.g., vitamin B12 and vitamin B2); active pharmaceutical ingredients (e.g., penicillin, cephalosporin, erythromycin oxytetracycline, tetracycline, demeclocycline, lincomycin, gentamycin potassium, and clavolanate).

In some embodiments, the cell or tissue culture or fermentation product includes one or more desired products as previously described. The cell or tissue culture product may include an elevated level of one or more desired products, as compared to a reference cell or tissue culture product. A “reference cell or tissue culture product” is a product produced under the same conditions as the cell or tissue product of the present disclosure is produced except using a reference culture medium as defined herein instead of a growth medium that comprises a biomass or derivative thereof of the C₁ metabolizing non-photosynthetic bacterium. In some embodiments, the cell or tissue culture product includes at least 5% more, at least 10% more, at least 15% more, at least 20% more, at least 25% more, at least 30% more, at least 35% more, at least 40% more, at least 45% more, or at least 50% more of the desired product than the reference cell or tissue culture product. In some embodiments, the cell or tissue culture product includes 5% to 200% more, 5% to 100% more, 5% to 50% more, 10% to 200% more, 10% to 100% more, 5% to 25% more, 25% to 50% more, 50% to 100% more, or 100% to 200% more of the desired product than the reference cell or tissue culture product.

In some embodiments, the desired product is a small molecule metabolite. A small molecule metabolite is a low molecular weight (e.g., up to 1500 daltons) organic compound, typically involved in a biological process as a substrate or product. Examples of small molecule metabolites include acetic acid, citric acid, lactic acid, isoascorbic acid, and glycerol. In particular embodiments, the small molecule metabolite is citric acid or lactic acid.

In some embodiments, the desired product is a vitamin. In particular embodiments, the vitamin is a B vitamin, such as vitamin B₆ or vitamin B₁₂.

In some embodiments, the desired product is an enzyme (e.g., a recombinantly produced enzyme). In particular embodiments, the enzyme is selected from chymosin, protease, lipase, amylase, cellulase, and carbohydrase. In particular embodiments, the desired product is an enzyme, and the cultured cells or tissue comprise a Bacillus species such as B. subtilis, E. coli, Corynebacterium glutamicum, or Pseudomonas putida.

Chymosin, also known as rennin, is a proteolytic enzyme that is capable of coagulating or curdling milk and commonly used in cheese making. Bovine chymosin is a commonly used form of chymosin and can be recombinantly produced. In certain embodiments, the desired product is a recombinantly produced chymosin.

Examples of proteases that may be produced as desired products include alcalases, savinases, esperases, papaine, Serine proteases, subtilisins, Aspartic proteases, pepsins, trypsins.

Examples of lipases that may be produced as desired products include plant derived lipases and animal derived lipases.

In some embodiments, the desired product is an omega-3 fatty acid. In particular embodiments, the omega-3 fatty acid is eicosapentaenoic acid (EPA) and/or ridocosahexaenoic acid (DHA). In certain embodiments, the omega-3 fatty acid is produced from a culture of marine dwelling bacteria or algal cells.

In some embodiments, the desired product is a carotenoid. Carotenoids are pigments derived from tetraterpenes, which are compounds consisting of 8 isoprene (C5) units comprising a 40-carbon polyene structure. Carotenoids that may be produced according to the present disclosure include astaxanthin, β-carotene, lutein, lycopene, antheraxanthin, fucoxanthin, diatoxanthin, diadinoxanthin, zeaxanthin, canthaxanthin). In certain embodiments, carotenoids are produced from a culture of microalga (e.g., Haematococcus pluvialis), bacteria (e.g., Paracoccus carotinifaciens), and yeast (e.g., Rhodotorula sp., Rhodosporidium sp., Sporobolomyces sp., Xanthophylomyces sp., Phaffia rhodozyma).In some embodiments, the desired product is xantham gum. Xantham gum is an edible polysaccharide that has a wide range of industrial uses, including foods, petroleum products, and cosmetics. In particular embodiments, the desired product is xantham gum, and the cultured cells or tissue comprise a Xanthamonas species such as Xanthomonas campestris.

In some embodiments, the desired product is a bio-based polymer. Bio-based polymers are defined as materials for which at least a portion of the polymer consists of material produced from renewable raw materials such as a plant or microorganism. Examples of bio-based polymers include polylactic acid biopolymer (PLA), the poly L-lactide, polyhydroxybuturate, polyhydroxyalkalonates (PHAs), poly-hydroxybutyrates (PHB) polyamide, polypropylene (PP). In particular embodiments, the desired product is a bio-based polymer, and the cultured cells or tissue comprise Pseudomonas putida. In particular embodiments, the bio-based polymer is a polyhydroxyalkalonate (PHA), and the cultured cells or tissue comprise Pseudomonas putida.

In certain embodiments, the desired product is one or more of the following: aromatics (e.g., vailin, o-creso, 4-hydroxyquinaldine, p-coumarate, p-hydroxystyrene, phenol, cinnamate, and anthranilate), dicarboxylic acids (e.g., muconates, adipate and furandicarboxylic acid), acids and alcohols (e.g., lactate, pyruvate, glycolate, glyoxylate, oxalate, acetate, ethanol, ethylene, and n-octanol), lactones (e.g., 4-valerolactone), glycolipids (e.g., rhamnolipids), terpenoids (e.g., β-carotene, zeaxanthin, and lycopene), natural products (e.g., myxochromide S, myxothiazol A, prodigiosin, 2,4-diacetylphloroglucinol, faviolin, tubulysin, phenazine carboxylate, and quinolones), fatty acids (e.g., docosahexaenoate), proteins (e.g., antibody fragments), biopolymers (e.g., PHAs), alginates, polyketides, and non-ribosomal peptides. In particular embodiments, the cultured cells or tissue comprise Pseudomonas putida.

In some embodiments, the cell or tissue culture or fermentation product includes an elevated concentration of omega-3 fatty acids as compared to a reference cell or tissue culture product. In some embodiments, the cell or tissue culture product includes at least 5% more, at least 10% more, at least 15% more, at least 20% more, at least 25% more, at least 30% more, at least 35% more, at least 40% more, at least 45% more, or at least 50% more of an omega-3 fatty acid than the reference cell or tissue culture product. In some embodiments, the cell or tissue culture product includes 5% to 100% more, 5% to 50% more, 10% to 200% more, 10% to 100% more, 5% to 25% more, 25% to 50% more, 50% to 100% more, or 100% to 200% more of the omega-3 fatty acid than the reference cell or tissue culture product.

In some embodiments, the desired product is a biomass, or a derivative thereof, of the cells or tissue that was cultured in the culture media.

In some embodiments, the desired product is heme iron. In some embodiments, the cell or tissue culture product includes an elevated level of heme iron or hemoprotein as compared to a reference cell or tissue culture product. In some embodiments, the cell or tissue culture product includes at least 5% more, at least 10% more, at least 15% more, at least 20% more, at least 25% more, at least 30% more, at least 35% more, at least 40% more, at least 45% more, or at least 50% more heme iron or hemoprotein than the reference cell or tissue culture product. In some embodiments, the cell or tissue culture product includes 5% to 100% more, 5% to 50% more, 10% to 200% more, 10% to 100% more, 5% to 25% more, 25% to 50% more, 50% to 100% more, or 100% to 200% more heme iron or hemoprotein than the reference cell or tissue culture product.

In some embodiments, the cell or tissue culture product includes an enhanced total level of iron as compared to a reference cell or tissue culture product. The iron in the cell or tissue culture product may be in the form of heme iron and/or in another form that is not coordinated to a porphyrin molecule in heme (non-heme iron). In some embodiments, the cell or tissue culture product includes at least 2% more, 5% more, at least 10% more, at least 15% more, at least 20% more, at least 25% more, at least 30% more, at least 35% more, at least 40% more, at least 45% more, or at least 50% more total iron than the reference cell or tissue culture product. In some embodiments, the cell or tissue culture product includes 5% to 100% more, 5% to 50% more, 10% to 200% more, 10% to 100% more, 5% to 25% more, 25% to 50% more, 50% to 100% more, or 100% to 200% more total iron than the reference cell or tissue culture product. Without wishing to be bound by any theory, the enhanced total level of iron is believed to be resulted at least partially from the enhanced level of heme iron provided by the C₁ metabolizing non-photosynthetic bacterium biomass or derivative thereof in the culture or fermentation medium.

In some embodiments, the cell or tissue culture product includes an enhanced level of non-heme iron as compared to a reference cell or tissue culture product. In some embodiments, the cell or tissue culture product includes at least 2% more, 5% more, at least 10% more, at least 15% more, at least 20% more, at least 25% more, at least 30% more, at least 35% more, at least 40% more, at least 45% more, or at least 50% more non-heme iron than the reference cell or tissue culture product. In some embodiments, the cell or tissue culture product includes 5% to 100% more, 5% to 50% more, 10% to 200% more, 10% to 100% more, 5% to 25% more, 25% to 50% more, 50% to 100% more, or 100% to 200% more non-heme iron than the reference cell or tissue culture product. Without wishing to be bound by any theory, the enhanced level of non-heme iron is believed to be resulted at least partially from the enhanced level of heme iron provided by the C₁ metabolizing non-photosynthetic bacterium biomass or derivative thereof in the culture or fermentation medium.

In some embodiments, the biomass or derivative thereof of the hemoprotein-producing C₁ metabolizing non-photosynthetic bacterium comprises an autolysate, and wherein the biomass of the cultured cells or tissue comprises an elevated level of heme or hemoprotein as compared to a biomass of cells or tissue cultured in a reference culture medium that does not comprises the autolysate. In some embodiments, the biomass of the cells or tissue comprises a cell-based meat product or a meat alternative product.

In some embodiments, the cell or tissue culture product includes an elevated level of iron, heme, and/or hemoprotein and is selected from: a culture product of mushroom cells or tissue, a probiotic, a dairy culture, a meat-curing culture, a vegetarian meat alternative product, or a combination thereof. Cell or tissue culture products having an elevated level of heme may have desired properties such as enhanced flavor, and/or enhanced visual appeal. Enhanced flavors of the cell or tissue culture products having an elevated level of heme may include more umami flavor and/or more metallic flavor. Enhanced visual appeal of cell or tissue culture products having an elevated level of heme may include a redder color. Enhanced flavor and enhanced visual appeal may be assessed by a panel of taste-testers.

In some embodiments, the cell or tissue culture product exhibits an isotopic δ ¹³C value lower than that of a reference cell or tissue culture product. The lower isotopic δ ¹³C value is based on the presence of the biomass of the C₁ metabolizing non-photosynthetic bacterium or derivative thereof in the culture medium during culture of the cells or tissue of the culture product and the absence of the biomass C₁ metabolizing non-photosynthetic bacterium or derivative thereof in the culture medium during culture of the cells or tissue of the reference cell or tissue product. In some embodiments, the isotopic δ ¹³C value is at least 1% lower, at least 2% lower, at least 3% lower, at least 4% lower, at least 5% lower, 6% lower, at least 7% lower, at least 8% lower, at least 9% lower, at least 10% lower than that of the reference culture product. In some embodiments, the isotopic δ ¹³C value is 1% lower to 50% lower, 5% lower to 50% lower, 1% lower to 5% lower, or 5% lower to 10% than that of the reference culture product.

In further embodiments, the cell or tissue culture product exhibits an isotopic δ ¹⁵N value lower than that of the reference cell or tissue culture product. The lower isotopic δ ¹⁵N value is based on the presence of the biomass of the C₁ metabolizing non-photosynthetic bacterium or derivative thereof during culture of the cells or tissue of the culture product and the absence of the biomass of C₁ metabolizing non-photosynthetic bacterium or derivative thereof in the culture medium during culture of the cells or tissue of the reference cell or tissue product. In some embodiments, the isotopic δ ¹⁵N value is at least 1% lower, at least 2% lower, at least 3% lower, at least 4% lower, at least 5% lower, 6% lower, at least 7% lower, at least 8% lower, at least 9% lower, at least 10% lower than that of the reference culture product. In some embodiments, the isotopic δ ¹⁵N value is 1% lower to 50% lower, 5% lower to 50% lower, 1% lower to 5% lower, or 5% lower to 10% than that of the reference culture product.

In further embodiments, the cell or tissue culture product exhibits an isotopic δ ³⁴S value lower than that of the reference cell or tissue culture product. The lower isotopic δ ³⁴S value is based on the presence of the biomass of the C₁ metabolizing non-photosynthetic bacterium or derivative thereof in the culture medium during culture of the cells or tissue of the culture product and the absence of the biomass of C₁ metabolizing non-photosynthetic bacterium or derivative thereof in the culture medium during culture of the cells or tissue of the reference cell or tissue product. In some embodiments, the isotopic δ ³⁴S value is at least 1% lower, at least 2% lower, at least 3% lower, at least 4% lower, at least 5% lower, 6% lower, at least 7% lower, at least 8% lower, at least 9% lower, at least 10% lower than that of the reference culture product. In some embodiments, the isotopic δ 34S value is 1% lower to 50% lower, 5% lower to 50% lower, 1% lower to 5% lower, or 5% lower to 10% than that of the reference culture product.

In some embodiments, the cell or tissue culture product is a bacterial cell product. Examples of bacterial cell products include phytoprotective bacterial cell products, probiotics, dairy-making cultures, meat-curing cultures, and bacterial cultures for alcoholic beverage fermentation.

In some embodiments, the cell or tissue culture product comprises a phytoprotective bacterial cell product. A phytoprotective bacterial cell product may be in the form of a liquid culture of the bacterial cells that is directly applied to a plant.

In some embodiments, the cell or tissue product is a non-bacterial cell or tissue product. The non-bacterial cell or tissue product may be an algal cell product, a fungal cell or tissue product, or an animal cell or tissue product.

Algal cell products may include, for example, algal oils that contain one or more omega-3 fatty acids, such as eicosapentaenoic acid (EPA) and/or docosahexaenoic acid (DHA), or algal carotenoids, such as astaxanthin, zeaxanthin, lutein, antheraxanthin, fucoxanthin, diatoxantin, and diadinoxanthin.

Fungal cell or tissue products include yeast cell products and mushroom cell or tissue product. As one example, fungal cell or tissue products may be used as vegetarian meat alternative food products. “Vegetarian meat alternative product” or “meat alternative product” refers to a food product that is derived from a non-animal organism but has meat-like qualities such as a meat-like flavor and/or a meat-like appearance (such as a red or reddish brown color). Vegetarian meat alternative products, for example, may be produced from yeast cells or mushroom cells or tissue, and may have meat-like properties that are conferred by culturing the yeast cells or mushroom cells or tissue with a biomass of the C₁ metabolizing non-photosynthetic bacterium. For example, elevated heme iron in the cell or tissue culture product may provide a meat-like flavor and/or a meat-like appearance to fungal cells or tissue cultured in a growth medium that includes a biomass or derivative thereof of C₁ metabolizing non-photosynthetic bacterium. Meat-like flavor and meat-like appearance may be assessed by a panel of taste-testers.

In certain embodiments, the cell or tissue product is a non-microorganism cell or tissue product. The non-microorganism cell or tissue product may be a non-microorganism algal, fungal, or animal cell or tissue product. Microorganisms are microscopic organisms, which may exist in their single-celled form or in a colony of cells. The non-microorganism cell or tissue product is a product produced from cell or tissue culture of an organism other than microorganisms.

Animal cell or tissue products may include fish, avian, insect, and mammalian cell or tissue culture products. Animal cell or tissue products may include cell-based meat products. Cell-based meat products are meat products that are produced from animal cell or tissue culture, rather than harvested from live animals.

In some embodiments, the cell or tissue products are processed to produce food products and food ingredients. Methods of making the food products and food ingredients are provided herein. In some embodiments, the methods include producing a cell culture product by the culture methods as described herein, and processing the cell culture product to produce the food product or food ingredient. Processing the cell culture product may include separation, filtration, clarification, precipitation, flocculation, evaporation, and/or drying of the cell culture product.

In some embodiments, the food product or food ingredient is a yeast product. In some embodiments, the yeast product is a starter culture for fermented beverage production, such as for kefir or kombucha or for an alcoholic beverage (e.g., beer and wine). In some embodiments, the starter culture is a beer starter culture or a wine starter culture. In some embodiments, the yeast product is baker's yeast or nutritional yeast.

In some embodiments, the food product or food ingredient is a bacterial cell product. In some embodiments, the bacterial cell product is a dairy-making culture such as a yogurt-making culture, a cheese-making culture, or an alcoholic beverage culture such as for sour beer. Bacterial cultures used for cheese-making include Lactococcus, Lactobacillus, and Streptococcus. Bacterial cultures used for yogurt making include Streptococcus thermophilus and Lactobacillus bulgaricus. Bacterial cultures for alcoholic beverage fermentation include Lactobacillus and Pediococcus. In some embodiments, the bacterial cell product is a meat-curing culture. Examples of bacteria involved in meat curing include Pediococcus cerevisiae, Micrococcus, Leuconostoc, certain Staphylococcus species, Lactobacillus, and Penicillium nalgiovense.

In some embodiments, the food product or food ingredient is a probiotic. In some embodiments, the probiotic is produces from a bacterial cell culture product. In some embodiments, the bacterial cells in the probiotic include one or more of: Lactobacillus acidophilus, Lactobacillus casei, Lactobacillus fermentum, Lactobacillus gasseri, Lactobacillus johnsonii, Lactobacillus paracasei, Lactobacillus plantarum, Lactobacillus rhamnosus, Lactobacillus salivarius, Bifidobacterium adolescentis, Bifidobacterium animalis, Bifidobacterium bifidum, Bifidobacterium breve, and Bifidobacterium longum.

In some embodiments, the desired product is a small molecule, an alcohol, an enzyme, or a bio-based polymer, and the culture cells comprise P. putida.

In some embodiments, the food product or food ingredient is a flavorant and/or fragrance and or a preservative such as vanillin, menthol, nootkatone, valencene, patchouli, and vetiver. In some embodiments, the food product or food ingredient is a preservative such as lactic acid, rosmarinic, or carnosic acid.

### EXAMPLES

### Example 1

### Production of a Methylococcus Capsulatus Bath (NCIMB 11132) Biomass AUTOLYSATE

Methylococcus capsulatus Bath and heterotrophic bacteria Cupriavidus sp. DB3 (strain NCIMB 41527), Aneurinibacillus sp. DB4 (strain NCIMB 41528) and Brevibacillus agri DB5 (strain NCIMB 41525) were cultured in a 5L stir tank by continuous aerobic fermentation of natural gas in an ammonium/mineral salts medium (AMS) at 45° C., pH 6.5. The AMS medium contains the following per litre: 10 mg NH₃, 75 mg H₃PO₄·2H₂O, 380 mg MgSO₄·7H₂O, 100 mg CaCl₂·2H₂O, 200 mg K₂SO₄, 75 mg FeSO₄·7H₂O, 1.0 mg CuSO₄·5H₂O, 0.96 mg ZnSO₄·7H₂O, 120 μg CoCl₂·6H₂O, 48 μg MnCl₂·4H₂O, 36 μg H₃BO₃, 24 μg NiCl₂·6H₂O and 1.20 μg NaMoO₄·2H₂O. The fermentor was filled with water which had been heat-sterilized at 125° C. for 10 secs. Addition of the different nutrients were regulated according to their consumption. Continuous fermentation was operated with 1-3% biomass (on a dry weight basis). The biomass was subjected to centrifugation in disc stack centrifuge at 3,000 rpm, optionally followed by ultrafiltration using membranes having an exclusion size of 100, 000 Daltons. The resulting product was then subjected to homogenization in an industrial homogenizer (pressure drop: 1000 bar (100 MPa); inlet temperature: 15° C. to produce a homogenized biomass (sample B145)). The 10 to 18% suspension of biomass was heated to the optimum reaction temperature of 50 to 55° C. and the pH is adjusted to 7.0-7.5 by the addition of NaOH. Incubation time was 45 minutes to 3 hours during which time the temperature of the material was kept within the range of from 50 to 55° C. and the pH was maintained in the optimal range of from 7.0 to 7.5 for 3 hours (sample B135). For the more soluble autolysate samples (samples B139, B140 and B141 generated by incubation of 1, 2, and 3 hours, respectively), a protease (serine endopeptidase cocktail that consists primarily of subtilisin A) was added to the homogenized biomass to increase the degree of hydrolysis at the beginning of the incubation step. Less soluble samples were produced in the absence of the protease. Following incubation the biomass was subjected to a heat inactivation step at 70 to 80 degrees Celsius for 1 to 5 minutes. The samples were then freeze dried (in some instances, the samples could be spray dried).

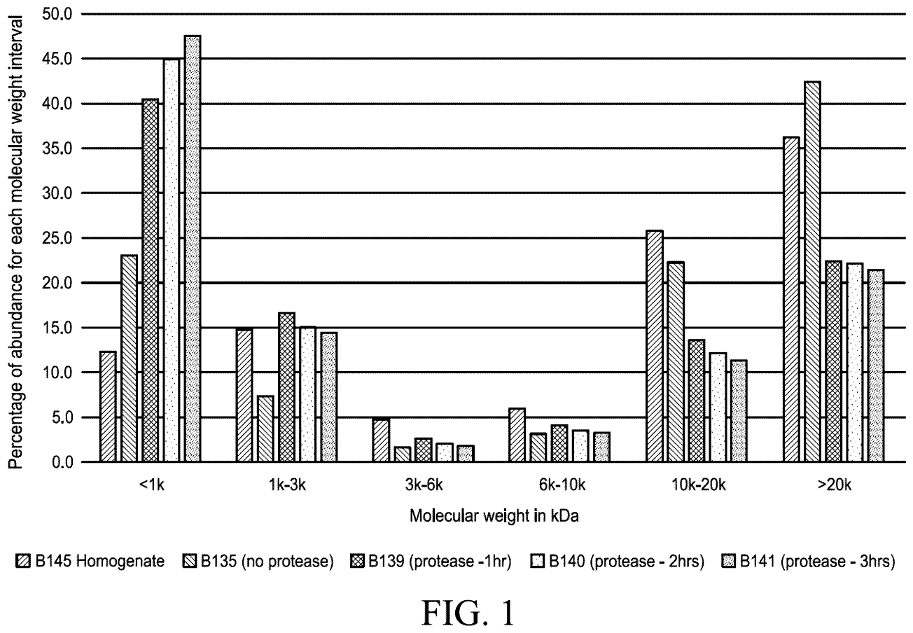

The molecular weight distribution of 4 autolysate samples (more soluble autolysate samples B139, B140 and B141 obtained by adding a protease to the autolysis step, and a less soluble autolysate sample, B135, no protease addition) and a homogenate sample (B145, which was processed equivalently up to the step of producing a homogenized biomass, but with no autolysis step) was analyzed by High-performance liquid chromatography (HPLC) and Gel permeation chromatography (GPC) using a TSKgel® G2000SWXL Size Exclusion column from Tosoh Bioscience. The results are shown in FIG. 1. The molecular weight distribution shows that all autolysate samples have relatively more small size peptides (more than 20% peptides are <1 kDa) than the homogenate sample (only about 12% peptides are below 1 kDa). This increased level of autolysis is even more visible in samples treated with the protease (>40% peptides are <1kDa)—the longer the incubation time, the more small peptides there are.

The characteristics of additional batches (B143, B149, B146 and B152) of the autolysate were analyzed and the summary is shown in Table 1.

TABLE 1

| | | | | | | Free |
|---|---|---|---|---|---|---|
| | | | | | | Amino |
| | | | | | | acids |
| | Incubation | | Crude | | Protein | (% of |
| Batch | Time | With | protein | % | Solubility | total |
| No. | (min) | protease | (%) | moisture | (%) | AA) |
| B143 | 180 | No | 62.2 | 10.4 | 70.5 | 38 |
| B149 | 90 | No | 64.5 | 9.4 | 58.9 | 38.5 |
| B146 | 60 | Yes | 65.9 | 5.14 | 77.6 | 42.4 |
| B152 | 45 | Yes | 67.4 | 3.9 | 80.4 | 47.5 |

Heme concentrations of seven autolysate samples (B137, B143, B149, B156, B146, B152, and B153) were measured by a method based on the conversion of heme to the fluorescent porphyrin derivative by removal of the heme iron under acidic conditions (Sassa S (1976) Sequential induction of heme pathway enzymes during erythroid differentiation of mouse Friend leukemia virus-infected cells. The Journal of experimental medicine 143(2):305-315). Heme iron was calculated using the relationship of 1 mole of heme iron/mole of heme. The heme and heme iron concentrations for the autolysate samples are shown in Table 2. Product types labelled “autolysate” in Table 2 refer to less soluble autolysates produced in the absence of any added protease. Product types labelled “autolysate HS” in Table 2 refer to more soluble lysates produced using an added protease, alcalase.

TABLE 2

| | | Incubation | mg heme/g | mg heme |
|---|---|---|---|---|
| Batch | Product Type | Time (min) | protein | iron/g protein |
| B137 | autolysate | 180 | 0.200 | 0.0181 |
| B143 | autolysate | 180 | 0.451 | 0.0408 |
| B149 | autolysate | 90 | 0.400 | 0.0359 |
| B156 | autolysate | 90 | 0.260 | 0.0236 |
| B146 | autolysate HS | 60 | 0.356 | 0.0322 |
| B152 | autolysate HS | 45 | 0.360 | 0.0327 |
| B153 | autolysate HS | 90 | 0.310 | 0.0281 |

HS = high solubility

### Example 2

### Growth of Marine Dwelling Organisms Cultured with a Biomass of a C₁ Metabolizing Non-Photosynthetic Bacterium

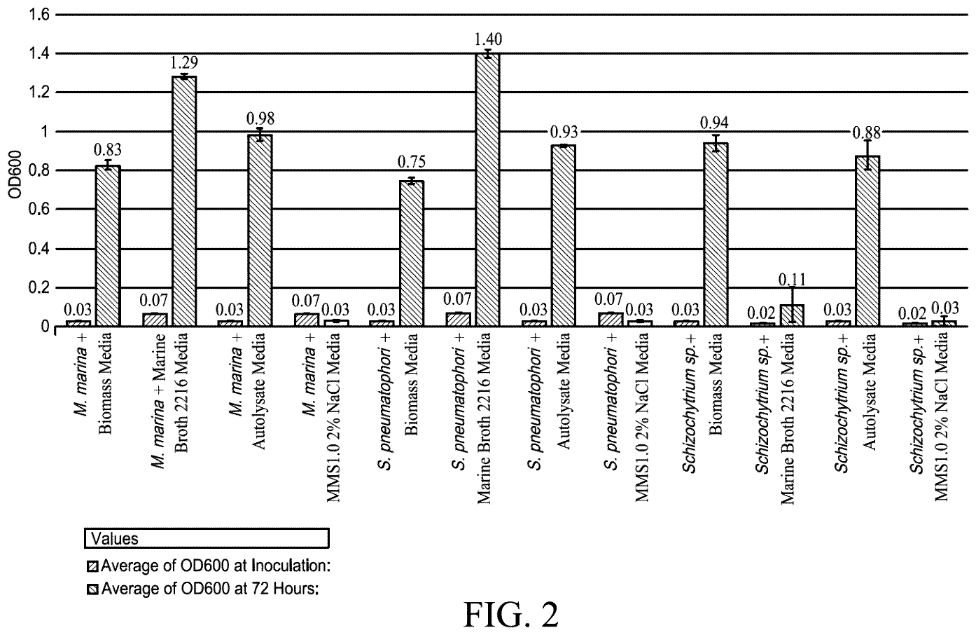

In the following example, Moritella marina, Shewanella pneumatophori, and Schizochytrium sp. ATCC 20888 were cultured in (a) (1) a culture medium including a biomass of Methylococcus capsulatus Bath or (2) a culture medium including an autolysate produced from the biomass, (b) a culture medium with carbon source but without any Methylococcus capsulatus Bath biomass or autolysate, or (c) a culture medium without carbon source (as a negative control). The organisms were first grown in an autoclaved and filtered Difco™ Marine Broth 2216 in baffled flasks at 140 RPMs, 19° C., for 48 hours. Cells were then washed three times in MMS1.0+2% NaCl to remove the pre-existing media. The biomass of Methylococcus capsulatus Bath was obtained as in Example 1, suspended, autoclaved, centrifuged, and filtered, and added to a culture broth including 1× MMS1.0 and 2% NaCl, with a final biomass concentration of 6.31 mg/ml as measured by spectrophotometry at A₂₈₀ₙₘ. The washed cells were used to inoculated three 2.5 ml culture flasks including the culture media with the biomass, and three 2.5 ml culture flasks including a negative control broth of 1× MMS1.0 and 2% NaCl, without a carbon source. Time points were recorded every 24 hours for 48 hours to monitor growth. As shown in FIG. 2, all three organisms grew well in the culture media including the biomass, but failed to grow in the negative control broth.

### Example 3

### Omega-3 Fatty Acid Production from Cells Cultured with a Biomass of a C₁ Metabolizing Non-Photosynthetic Bacterium

In the following example, Moritella marina, Shewanella pneumatophori, and Schizochytrium sp. were analyzed for production of omega-3 fatty acids following culture of the cells in various culture media as described below.

To generate autolysate, 2.5 grams of wet cell pellet of wild-type M. capsulatus Bash was lysed via heavy sonication in 20 mls of MMS1.0. Next, the unclarified lysate was incubated at 54-58° C., pH=7.0, for 3 hours, to produce an autolysate. The NaCl concentration was then increased from 0.2% to 2% with sterile 5M NaCl followed by a rigorous clarification and sterile filtration.

100 ul from the S. pneumatophori or M. marina and 200 ul of Schizochytrium sp. culture was used to inoculate each of: (i) three 2.5 mls flasks of culture medium including the autolysate, MMS1.0 and 2% NaCl (“autolysate media”), (ii) three 2.5 ml flasks of culture medium including the biomass, MMS1.0 and 2% NaCl (“biomass media”), (iii) three 2.5 mls flasks of Marine Broth (as a positive control), and (iv) three 2.5 mls of MMS1.0 and 2% NaCl with no carbon source (as a negative control). The remaining seed culture replicates were consolidated and submitted for FA analysis as time point 0, and the dilution factor of the inoculated secondary cultures was taken into account.

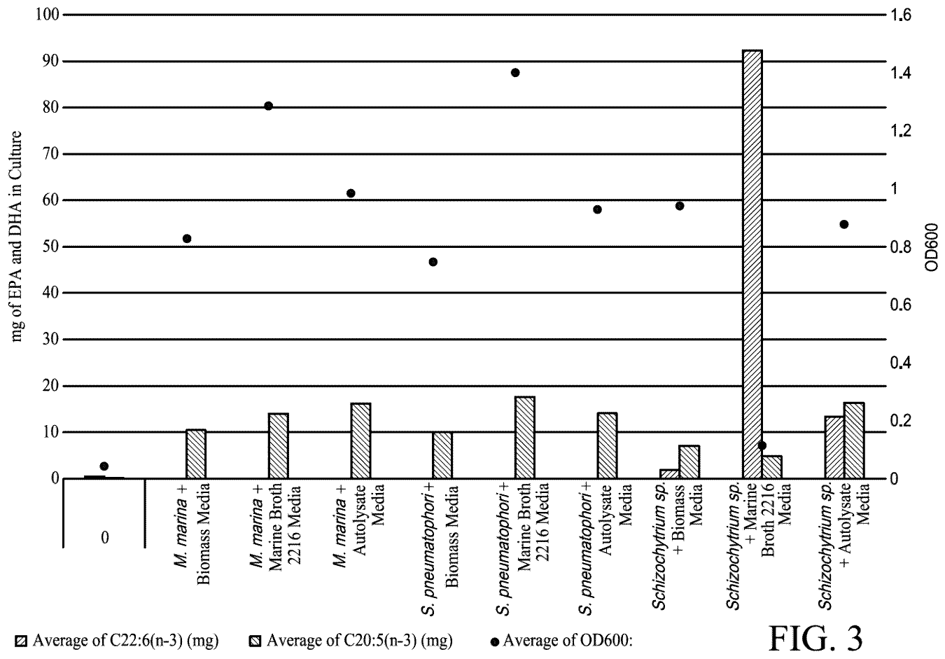

These cultures were allowed to grow at 19° C. and 160 RPMs for 72 hours prior to sampling. As expected, the negative controls showed no growth and thus were not submitted for fatty acid analysis. All other cultures showed growth but relatively low biomass and thus, all replicates from each conditions were consolidated into a single sample for submission for GC-mass spectrometry analysis. Production of DHA (C22:6(n-3)) and EPA (C20:5(n-3)) were measured and the results are shown in FIG. 3. As shown in FIG. 3, EPA was produced in all cultures, and DHA was produced in the Schizochytrium sp. cultures. The results thus demonstrated that omega-3 fatty acids can be produced by marine dwelling microorganisms cultured in the presence of a biomass or autolysate thereof of a C₁ metabolizing non-photosynthetic bacterium.

### Example 4

### Growth of Industrially Relevant Microorganisms Cultured with a Biomass of a C₁ Metabolizing Non-Photosynthetic Bacterium

In the following example, Bacillus licheniformis (ATCC 53757), Escherichia coli (ATCC 25922), Lactobacillus reuteri (DSM 20053), and Pichia jadini (CBS 4511) were each cultured in (1) a culture medium including an autolysate produced of Methylococcus capsulatus Bath biomass, (2) a culture medium including an autolysate produced with additional protease from the biomass, or (3) a culture medium with carbon source but without any Methylococcus capsulatus Bath autolysate. The autolysate concentrations were based on the nitrogen (N) content of the autolysates (Table 3) and ranged from 0.03-1 g/L N. For the growth of L. reuteri, the autolysates were the sole source of nitrogen. Stock solutions of the autolysates were made (5 g/L N) and autoclaved. The sterile autolysates were added to sterile base media at the concentrations listed in Table 4.

TABLE 3
Production process and analytical data for the autolysates.

| | Autolysis | | | Crude | Protein | Free aa |
|---|---|---|---|---|---|---|
| | time | | Inactivation | protein | solubility | [% of |
| Hydrolysate | [min] | Protease | (temp/time) | [%] | [%] | total aa] |
| B199 | 180 | No | 70°/60 min | 65.1 | 60.1 | 38.8 |
| B223 | 90 | Yes | 80°/1 min | 66.7 | 81.7 | 46.4 |

TABLE 4
Concentrations of sterile autolysates added to sterile base media

| | Concentration of sterile |
|---|---|
| | autolysates in culture media |
| Microorganism | (g/l N) |
| Bacillus licheniformis | 0.03, 0.06, 0.1 |
| Pichia jadini | 0.03, 0.06, 0.1 |
| Lactobacillus reuteri* | 0.2, 0.6, 1.0 |
| E. coli | 0.11, 0.22 |

*For comparison to media without autolysate, MRS (Oxoid) was used.

The cultures were grown in a micro-reactor system (Bio-Lector, Beckman) that allowed the screening of multiple parallel microfermentations under controlled conditions (pH, dissolved oxygen). The cultivation conditions are listed in Table 5. The BioLector measures cell density via sensors based on a principle other than optical density (OD). The cultures were incubated for 20-30 hrs and cell density, dissolved oxygen, and pH measurements were recorded at regular intervals.

TABLE 5
BioLector cultivation conditions

| | B. lich., C. glut., | | |
|---|---|---|---|
| Parameter | P. jadini | L. reuteri | E. coli |
| Plate type | Flower | Round well | Flower |
| Liquid volume [ml] | 1.0 | 1.2 | 0.83 |
| pH | 6.8 | 6.5 | 6.6 |
| Stirring [rpm] | 1000 | 400 | 1400 |
| Temperature [° C.] | 37 | 30 | 37 |

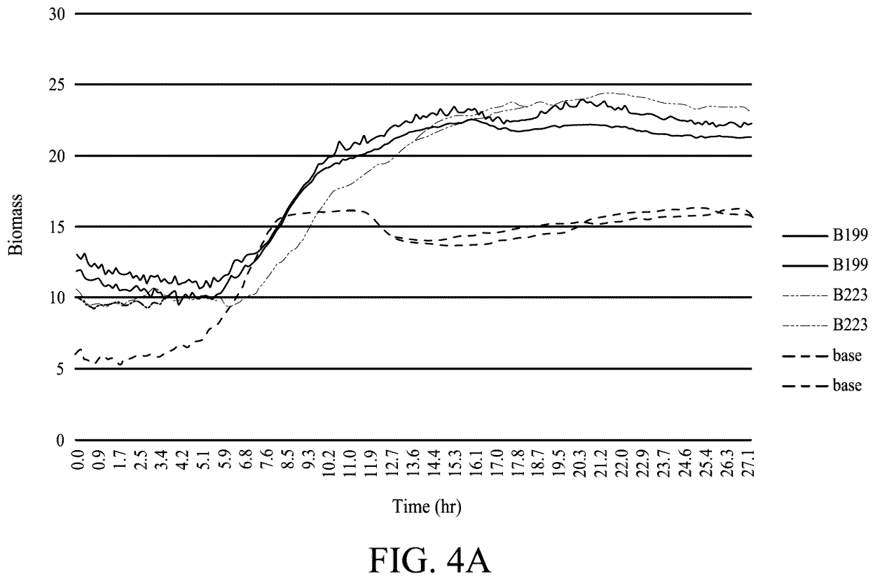

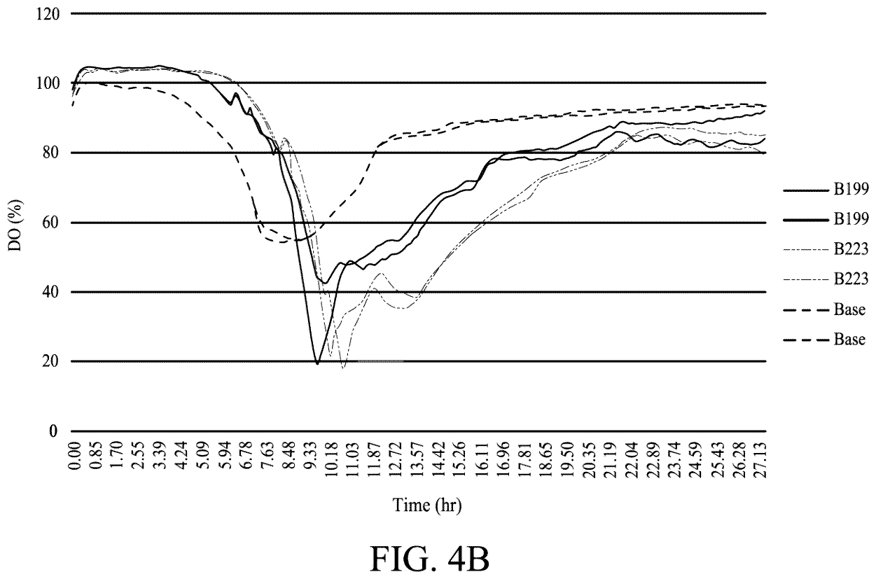

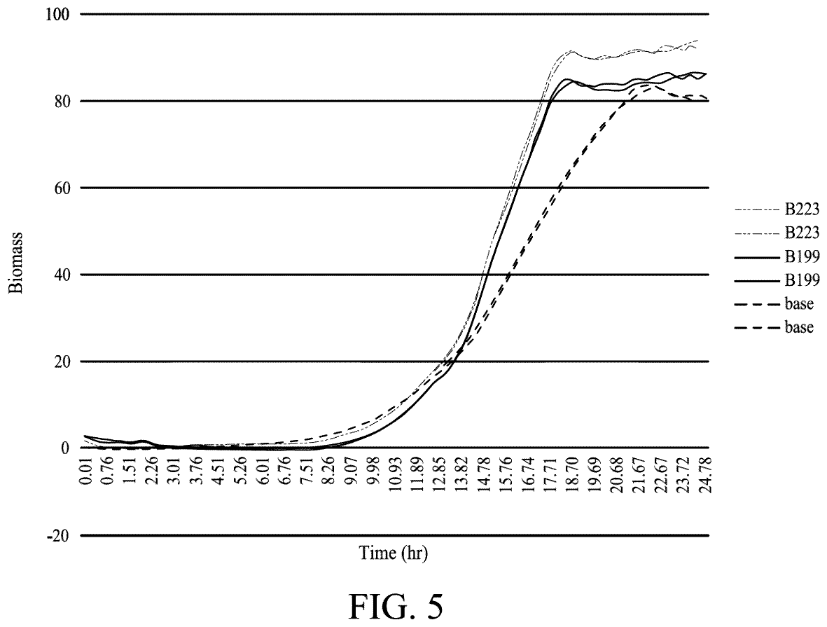

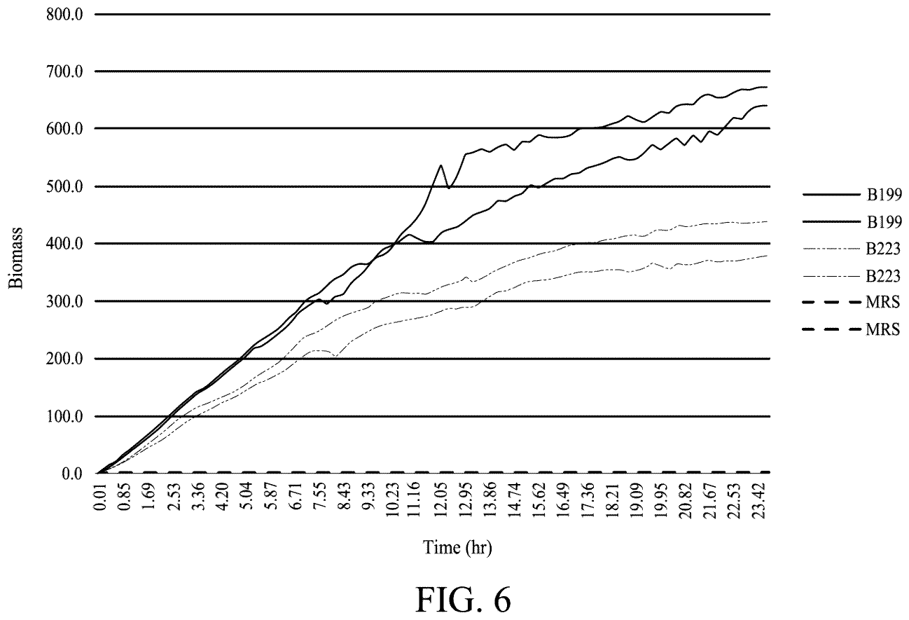

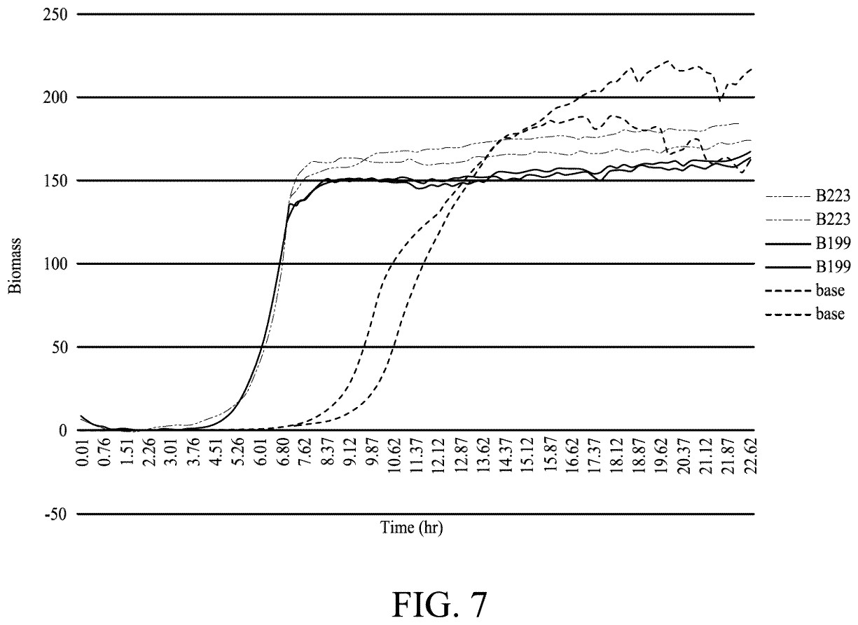

The autolysates were not completely soluble, however, despite high background readings at the higher concentrations used, the increase in biomass was readily detectable. As shown in FIGS. 4-7, the autolysates supported growth and were a source of nutrients for each strain.

While specific embodiments of the invention have been illustrated and described, it will be readily appreciated that the various embodiments described above can be combined to provide further embodiments, and that various changes can be made therein without departing from the spirit and scope of the invention.

All of the U.S. patents, U.S. patent application publications, U.S. patent applications, foreign patents, foreign patent applications and non-patent publications referred to in this specification, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

That which is claimed is:

1. A method of cell or tissue culture or fermentation comprising culturing cells or tissue in a culture or fermentation medium that comprises a biomass or derivative thereof of a hemoprotein-producing C₁ metabolizing non-photosynthetic bacterium,
wherein the amount of heme in the biomass or derivative thereof is in the range of from 0.01 to 10 mg/g protein of the biomass or derivative thereof, and/or
wherein the amount of the biomass or derivative thereof in the culture or fermentation medium is in the range of 0.1 to 50 g/l.

2. The method of claim 1, wherein the cells or tissue comprises bacterial cells.

3. The method of claim 2, wherein the bacterial cells comprise Bacillus subtilis, Bacillus licheniformis, Escherichia coli, Lactobacillus reuteri, Corynebacterium glutamicum, Pseudomonas putida or Xanthomonas sp.

4. The method of claim 2, wherein the bacterial cells comprise Bifidobacterium lactis, Bf. bifidum, Bf. longum, Bf. infantis, Bf. animalis, Bf. breve, Saccharomyces boulardii, Streptococcus Thermophilus, or Bacillus Coagulans.

5. The method of claim 1, wherein the bacterial cells comprise marine dwelling bacterial cells.

6. The method of claim 1, wherein the cells or tissue comprises non-bacterial cells or tissue.

7. The method of claim 1, wherein the cells or tissue comprise algal cells, and optionally the culture or fermentation medium further comprises seawater base.

8. The method of claim 7, wherein the algal cells comprise microalgae.

9. The method of claim 7, wherein the algal cells comprise macroalgae.

10. The method of claim 1, wherein the cells or tissue comprise fungal cells or tissue.

11. The method of claim 10, wherein the fungal cells or tissue comprise yeast cells.

12. The method of claim 10, wherein the fungal cells or tissue comprise mushroom cells or tissue, and optionally the culture or fermentation medium further comprises potato extract, grain, and/or fruiting substrate.

13. The method of claim 1, wherein the cells or tissue comprise animal cells or tissue, and optionally the culture or fermentation medium further comprises serum, a growth hormone, a growth factor, an antibacterial agent and/or an antifungal agent.

14. The method of claim 12, wherein the animal cells or tissue comprise fish or shellfish cells or tissue, and optionally the culture or fermentation medium further comprises fish serum.

15. The method of claim 12, wherein the animal cells or tissue comprise avian cells or tissue.

16. The method of claim 1, wherein the animal cells comprise insect cells or tissue.

17. The method of claim 12, wherein the animal cells or tissue comprise mammalian cells or tissue.

18. The method of claim 17, wherein the animal cells comprise embryonic stem cells, adult stem cells, myosatellite cells, myoblasts, myocytes, and/or muscle cells.

19. The method of any one of claims 1 to 18, wherein the C₁ metabolizing non-photosynthetic bacterium comprises a methylotrophic bacterium.

20. The method of any one of claims 1 to 18, wherein the C₁ metabolizing non-photosynthetic bacterium comprises a methanotrophic bacterium.

21. The method of any claim 18, wherein the methanotrophic bacterium is selected from the group consisting of Methylomonas, Methylobacter, Methylococcus, Methylosinus, Methylocystis, Methylomicrobium, Methanomonas, and Methylocella.

22. The method of any of claims 20, wherein the methanotrophic bacterium comprises Methylococcus capsulatus.

23. The method of claim 20, wherein the methanotrophic bacterium comprises Methylococcus capsulatus (Bath).

24. The method of any one of claims 1 to 23, wherein the biomass of the hemoprotein-producing C₁ metabolizing non-photosynthetic bacterium or derivative thereof comprises a suspension, an isolate, an autolysate, an homogenate, a digestate, a hydrolysate, an isolate, an extract, or a combination thereof of the biomass.

25. The method of any one of claims 1 to 24, wherein the biomass or derivative thereof comprises essential amino acids at an amount within the range of 1-100 mg/g each.

26. The method of any one of claims 1 to 25, wherein the biomass or derivative thereof comprises copper at an amount within the range of 50-500 mg/kg.

27. The method of any one of claims 1 to 26, wherein the biomass or derivative thereof comprises iron at an amount within the range of 0.05 to 0.6 mg/g.

28. The method of any one of claims 1 to 27, further comprising separating the cultured cells from the growth medium to produce isolated culture cells and/or an isolated supernatant.

29. The method of any of one of claims 1 to 28, further comprising isolating, concentrating, separating or purifying a desired product from the fermented or cultured cells or tissue.

30. The method of claim 29, wherein the desired product is selected from vitamins, fatty acids, amino acids, nucleosides, peptides, proteins, enzymes, pigments, flavors, fragrances, organic acids, preservatives, small molecule metabolites, ferment, culture, probiotics, carotenoids, and meat alternatives.

31. The method of claim 29, wherein the desired product is a small molecule metabolite selected from citric acid and lactic acid.

32. The method of claim 29, wherein the desired product is an enzyme selected from chymosin, protease, lipase, amylase, cellulase, amylase, and carbohydrase.

33. The method of claim 29, wherein the desired product is an omega-3 fatty acid selected from eicosapentaenoic acid (EPA) and docosahexaenoic acid (DHA).

34. The method of claim 29, wherein the desired product comprises a biomass of cultured cells or tissue.

35. The method of claim 34, wherein the biomass of the cultured cells or tissue comprises a probiotic, a dairy culture, or a meat-curing culture.

36. The method of any of claims 1 to 35, wherein the biomass of the cultured cells or tissue comprises an elevated level of iron, heme or hemoprotein as compared to a biomass of cells or tissue cultured in a reference culture medium that does not comprise a biomass or derivative thereof of a hemoprotein-producing C₁ metabolizing non-photosynthetic bacterium.

37. The method of claim 36, wherein the biomass or derivative thereof of the hemoprotein-producing C₁ metabolizing non-photosynthetic bacterium comprises an autolysate of the hemoprotein-producing C₁ metabolizing non-photosynthetic bacterium.

38. The method of claim 37, wherein the cultured cells or tissue, biomass thereof, or derivative of the biomass is suitable for preparing a cell-based meat product or a meat alternative product.

39. The method of any of claims 1 to 38, wherein the culturing results in an improved growth rate, yield or productivity of the cells or tissue or the final target ingredient of the culture, as compared to culturing the cells or tissue in a reference culture medium that does not comprise a biomass or derivative thereof of a hemoprotein-producing C₁ metabolizing non-photosynthetic bacterium.

40. A cell or tissue culture or fermentation medium, comprising biomass or derivative thereof of a hemoprotein-producing C₁ metabolizing non-photosynthetic bacterium,
wherein the amount of heme in the biomass or derivative thereof is in the range of from 0.01 to 10.0 mg/g protein, and/or
wherein the amount of the biomass or derivative thereof in the culture medium is in the range of from 0.1 to 50 g/l.

41. The culture or fermentation medium of claim 40, wherein the amount of essential amino acids in the biomass or derivative thereof is in the range of from 1 to 100 mg/g each.

42. The culture or fermentation medium of claim 40 or 41, wherein the amount of bioavailable copper in the biomass or derivative thereof is in the range of from 50 to 500 mg/kg.

43. The culture or fermentation medium of any of claims 40 to 42, wherein the amount of heme is in the biomass or derivative thereof is in the range of from 0.1 to 1 mg/g protein of the biomass or derivative thereof.

44. The culture or fermentation medium of any of claims 40 to 43, wherein the culture or fermentation medium comprises a derivative of the biomass.

45. The culture or fermentation medium of claim 44, wherein the derivative is an autolysate, lysate, extract, isolate, suspension, homogenate, or digestate of the biomass of the hemoprotein-producing C₁ metabolizing non-photosynthetic bacterium.

46. The culture or fermentation medium of any of claims 40 to 45, wherein the culture or fermentation medium is suitable for bacterial cell culture.

47. The culture or fermentation medium of claim 46, wherein the culture or fermentation medium is suitable for culture of marine-dwelling bacteria.

48. The culture or fermentation medium of any of claims 40 to 45, wherein the culture or fermentation medium is suitable for non-bacterial cell culture.

49. The culture or fermentation medium of any one of 40 to 45, wherein the culture or fermentation medium is suitable for algal cell culture and optionally further comprises seawater base.

50. The culture or fermentation medium of any one of 40 to 45, wherein the culture or fermentation medium is suitable for fungal cell or tissue culture.

51. The culture or fermentation medium of claim 50, wherein the culture or fermentation medium is suitable for yeast cell culture.

52. The culture or fermentation medium of claim 50, wherein the culture or fermentation medium is suitable for mushroom cell or tissue culture and optionally further comprises potato extract, grain, and/or fruiting substrate.

53. The culture or fermentation medium of any one of claims 40 to 45, wherein the culture or fermentation medium is suitable for animal cell or tissue culture and optionally further comprises serum, a growth hormone, a growth factor, an antibacterial agent and/or an antifungal agent.

54. The culture or fermentation medium of claim 53, wherein the culture or fermentation medium is suitable for fish cell or tissue culture and optionally comprises fish serum.

55. The culture or fermentation medium of claim 53, wherein the culture or fermentation medium is suitable for avian cell or tissue culture.

56. The culture or fermentation medium of claim 53, wherein the culture or fermentation medium is suitable for insect cell or tissue culture.

57. The culture or fermentation medium of claim 53, wherein the culture or fermentation medium is suitable for mammalian cell or tissue culture.

58. The culture or fermentation medium of any one of claims 40 to 57, wherein the C₁ metabolizing non-photosynthetic bacterium is a methylotrophic bacterium.

59. The culture or fermentation medium of any one of claims 40 to 58, wherein the C₁ metabolizing non-photosynthetic bacterium is a methanotrophic bacterium.

60. The culture or fermentation medium of claim 59, wherein the methanotrophic bacterium is selected from the group consisting of Methylomonas, Methylobacter, Methylococcus, Methylosinus, Methylocystis, Methylomicrobium, Methanomonas, and Methylocella.

61. The culture or fermentation medium of claim 59, wherein the methanotrophic bacterium is Methylococcus capsulatus.

62. The culture or fermentation medium of claim 59, wherein the methanotrophic bacterium is Methylococcus capsulatus Bath.

63. A product of cell or tissue culture or fermentation produced by a method of any one of claims 1 to 39.

64. The product of claim 63, produced by the method of claim 28, wherein product comprises the isolated culture cells.

65. The product of claim 63, produced by the method of claim 28, wherein the product comprises isolated, purified or concentrated product of interest derived from the supernatant or the cells themselves.

66. The product of any one of claims 63 to 65, comprising an elevated level of one or more desired products, as compared to a reference product produced using a growth medium that does not comprise the biomass or derivative thereof of the C₁ metabolizing non-photosynthetic bacterium.

67. The product of claim 66, wherein the one or more desired products are selected from vitamins, fatty acids, amino acids, nucleosides, peptides, proteins, enzymes, pigments, flavors, fragrances, organic acids, preservatives, small molecule metabolites, ferment, culture, probiotics, and meat alternatives.

68. The product of claim 67, wherein the one or more desired products comprise a small molecule metabolite selected from citric acid and lactic acid.

69. The product of claim 67, wherein the one or more desired products comprise an enzyme.

70. The product of claim 69, wherein the enzyme comprises chymosin, protease, lipase, amylase, cellulase, and carbohydrase.

71. The product of any one of claims 63 to 70, wherein the desired product includes an elevated concentration of omega-3 fatty acids as compared to a reference product produced using a growth medium that does not comprise the biomass or derivative thereof of the C₁ metabolizing non-photosynthetic bacterium.

72. The product of claim 71, wherein the omega-3 fatty acids comprise eicosapentaenoic acid (EPA) and/or docosahexaenoic acid (DHA).

73. The product of any one of claims 63 to 72, wherein the product includes an elevated concentration of one or more carotenoids as compared to a reference product produced using a growth medium that does not comprise the biomass or derivative thereof of the C₁ metabolizing non-photosynthetic bacterium.

74. The product of claim 73, wherein the one or more carotenoids are selected from beta-carotene, lutein, lycopene, zeaxanthin, and astaxanthin.

75. The product of any one of claims 63 to 74, comprising an elevated level of iron, heme iron or hemoprotein as compared to a reference product produced using a growth medium that does not comprise the biomass or derivative thereof of the C₁ metabolizing non-photosynthetic bacterium.

76. The product of claim 75, comprising a cell-based meat product.

77. The product of any one of claims 63 to 76, comprising a probiotic, a dairy culture, or a meat-curing culture.

78. The product of any one of claims 63 to 76, comprising a vegetarian meat alternative product.

79. The product of any one of claims 63 to 76, comprising a phytoprotective bacterial cell product.

80. A product of non-bacterial cell or tissue culture or fermentation having a heme iron content that is higher than that of a reference product produced using a culture or fermentation medium that does not comprise biomass or derivative thereof of a C₁ metabolizing non-photosynthetic bacterium.

81. The product of claim 80, further comprising an isotopic δ ¹³C value lower than that of the reference product.

82. The product of claim 80 further having an isotopic δ ¹⁵N value lower than that of the reference product.

83. The product of claim 81 or 82 further having an isotopic δ ³⁴S value lower than that of the reference product.

84. The product of any one of claims 80 to 83, wherein the product is selected from non-microorganism cell or tissue culture or fermentation products.

85. The product of any one of claims 80 to 83, wherein the product is selected from products of algal, yeast, mushroom, fish, avian, insect, and mammalian cell or tissue culture or fermentation.

86. The product of claim 85, wherein the cell or tissue comprises a yeast or mushroom cell or tissue, and the product comprises a vegetarian meat alternative product.

87. The product of any one of claims 80 to 84, wherein the cell or tissue comprises an animal cell or tissue, and the product comprises a cell-based meat product.

88. A method of making a food product or ingredient, comprising: producing a product of cell or tissue culture or fermentation by a method of any one of claims 1 to 39, and processing the product to produce the food product or food ingredient.

89. The method of claim 88, wherein the processing the product comprises one or more of separation, filtration, clarification, precipitation, flocculation, evaporation, and drying.

90. A food product or food ingredient produced by a method of claim 88 or 89.

91. The food product or food ingredient of claim 90, wherein the cultured cell comprises yeast, and wherein the food product or food ingredient comprises a starter culture for fermented beverage production.

92. The food product or food ingredient of claim 91, wherein the food product or food ingredient comprises a starter culture for alcoholic beverage production.

93. The food product or food ingredient of claim 91, wherein the food product or food ingredient comprises a starter culture of beer, wine, kefir or kombucha production.

94. The food product or food ingredient of claim 91, wherein the food product or food ingredient comprises a vegetarian meat alternative product.

95. The food product or food ingredient of claim 94, wherein the cultured cell or tissue comprises fungal cells.

96. The food product or food ingredient of claim 90, wherein the cultured cells comprise bacterial cells, and wherein the food product or food ingredient comprises a dairy making culture or a meat-curing culture.

97. The food product or food ingredient of claim 96, wherein the food product or food ingredient comprises a yogurt making culture or a cheese making culture.

98. The food product of food ingredient of claim 90, wherein the cultured cells comprise bacterial cells, and wherein the food product or food ingredient comprises a probiotic.

99. The food product of food ingredient of claim 90, comprising a cell-based meat product.

100. The food product or food ingredient of any one of claims 90 to 99, comprising a cell-based meat, meat alternative product, amino acid, peptide, protein, fatty acid, organic acid, enzyme, pigment, flavor, fragrance, ferment, culture, probiotic, food ingredient, nucleoside, vitamin, small molecule, metabolite, flavorant, or preservative.
